SEQUENCE LISTING (31 sequences)

SEQ ID NO: 1 - DNA, 1539 bp, E. coli
  CDS (1)..(1539)
atggcttttgcctcattaattgaacgccagcgcattcgcctgctgctggcgttattattcggtgcctgcggaacgctggccttctctccttacgacgtctggcctgcggcgattatttcgctgatggggcttcaggcgttgacctttaaccgccgtccactccagtctgccgctattggcttttgctggggatttggcctctttggcagcggtattaactgggtctatgtcagcatcgcgacctttggcggaatgcctggcccggttaacatcttcctggtggtgctgctggcggcgtatttgtcgctgtataccggactgtttgctggcgtgctgtcgcgtctgtggccgaaaaccacctggctgcgcgtagcgattgccgcccctgccctctggcaagtgaccgagtttctgcgcggttgggtactgaccggcttcccgtggttacagttcggctatagccagattgatggcccgttaaaagggctggcaccgataatgggcgtggaagccattaacttcctgctgatgatggttagtggcctgctggcactggcgttggtcaaacgcaactggcgtccgctggtggtggccgtcgtgctgtttgcccttcccttcccgctgcgttacatccagtggtttaccccacaaccggagaaaaccattcaggtttcgatggttcagggcgatattccgcaatcgctgaaatgggacgaaggccagcttcttaatacgctgaagatttactacaacgcaacggcaccgctgatgggcaaatcatcgttgattatctggccggagtcggcgataaccgatctggaaattaatcagcaaccgttcctcaaagcactggacggtgagttgcgtgataaaggtagctcgctggtaaccgggattgtcgacgcgcgtctcaataagcagaaccgctacgatacctacaacaccatcatcacgctgggtaaaggtgcgccgtacagctacgaatcagccgatcgctataacaaaaaccatctggtgccgtttggcgagtttgtcccgctggagtcgattctgcgtccgttagcaccgttctttgatctgccgatgtcgtcgttcagccgtgggccatatatccagccgccgctgtcggcaaatggtattgagcttactgcggctatttgctacgagatcattctcggcgagcaagtgcgcgataacttccgcccggataccgactatctgctgactatctccaacgatgcgtggtttggtaaatctattggtccatggcaacacttccagatggcgcgaatgcgtgcgctggagctggcgcgcccactgttgcgcagcaccaacaacggcattacggcggtgattggcccgcagggtgagattcaggcgatgatcccgcagttcacccgcgaggtgttaaccactaacgtgacgccgaccaccggactcacaccatacgcacgtaccggcaactggccgctgtgggtgctgacggcattgtttggttttgctgctgtgttgatgagtctgcgtcagcgacgtaaataa

SEQ ID NO: 2 - protein, 512 aa, E. coli
MAFASLIERQRIRLLLALLFGACGTLAFSPYDVWPAAIISLMGLQALTFNRRPLQSAAIGFCWGFGLFGSGINWVYVSIATFGGMPGPVNIFLVVLLAAYLSLYTGLFAGVLSRLWPKTTWLRVAIAAPALWQVTEFLRGWVLTGFPWLQFGYSQIDGPLKGLAPIMGVEAINFLLMMVSGLLALALVKRNWRPLVVAVVLFALPFPLRYIQWFTPQPEKTIQVSMVQGDIPQSLKWDEGQLLNTLKIYYNATAPLMGKSSLIIWPESAITDLEINQQPFLKALDGELRDKGSSLVTGIVDARLNKQNRYDTYNTIITLGKGAPYSYESADRYNKNHLVPFGEFVPLESILRPLAPFFDLPMSSFSRGPYIQPPLSANGIELTAAICYEIILGEQVRDNFRPDTDYLLTISNDAWFGKSIGPWQHFQMARMRALELARPLLRSTNNGITAVIGPQGEIQAMIPQFTREVLTTNVTPTTGLTPYARTGNWPLWVLTALFGFAAVLMSLRQRRK

SEQ ID NO: 3 - protein, 6 aa, E. coli
FSPYDV

SEQ ID NO: 4 - protein, 12 aa, E. coli
YVSIATFGGMPG

SEQ ID NO: 5 - protein, 39 aa, E. coli
QWFTPQPEKTIQVSMVQGDIPQSLKWDEGQLLNTLKIYY

SEQ ID NO: 6 - DNA, 23 bp, Artificial
  5′ kan primer
gaattctacgtgcccgggagatc

SEQ ID NO: 7 - DNA, 26 bp, Artificial
  kan 3′ primer
taatgacccgggcgtgatgcggccgc

SEQ ID NO: 8 - DNA, 24 bp, Artificial
  5′ para primer
tctagactgtaacaaagcgggacc

SEQ ID NO: 9 - DNA, 24 bp, Artificial
  para 3′ primer
cccgtttttttgggctaggtcgac

SEQ ID NO: 10 - DNA, 64 bp, Artificial
  5′ ybe-k primer
gtcaaaatcccggatgactcaccccagccgaagctggatgaataagcggccgcatcacgcccgg

SEQ ID NO: 11 - DNA, 70 bp, Artificial
  p-lnt-3′ primer
cccgtttttttgggctaggtcgacccgaaactggatagataactacatggcttttgcctcattaattgaa

SEQ ID NO: 12 - DNA, 26 bp, Artificial
  5′ cutE primer
gaattcaccgaaactggatagataac

SEQ ID NO: 13 - DNA, 45 bp, E. coli
ctgcgtcagcgacgtaaacatcaccatcaccatcattaaggatcc

SEQ ID NO: 14 - DNA, 26 bp, Artificial
  5′-lnt primer
gaattcgaataacagcaatagtggac

SEQ ID NO: 15 - DNA, 46 bp, Artificial
  lnt-3′ primer
tctgcgacaacgacggaaacatcaccatcaccatcattaaggatcc

SEQ ID NO: 16 - DNA, 30 bp, Artificial
  5′-cutE435 primer
gaatgcgtgcgctgaagctggcgcgcccac

SEQ ID NO: 17 - DNA, 28 bp, Artificial
  5′-phoA primer
ctgcagctcagggcgatattactgcacc

SEQ ID NO: 18 - DNA, 28 bp, Artificial
  phoA-3′ primer
gccgctctggggctgaaataataagctt

SEQ ID NO: 19 - DNA, 27 bp, Artificial
  5′-lacZ primer
ctgcagccgtcgttttacaacgtcgtg

SEQ ID NO: 20 - DNA, 33 bp, Artificial
  lacZ-3′ primer
taccagttggtctggtgtcaaaaataaaagctt

SEQ ID NO: 21 - DNA, 26 bp, Artificial
  5′-lntEC primer
tctagaaccgaaactggatagataac

SEQ ID NO: 22 - DNA, 21 bp, Artificial
  lntEc30-3′ primer
cgctggccttctctcctgcag

SEQ ID NO: 23 - DNA, 23 bp, Artificial
  lntEc53-3′ primer
gacctttaaccgccgtcctgcag

SEQ ID NO: 24 - DNA, 23 bp, Artificial
  lntEc80-3′ primer
ggtctatgtcagcatcgctgcag

SEQ ID NO: 25 - DNA, 22 bp, Artificial
  lntEc117-3′ primer
ctgtcgcgtctgtggcctgcag

SEQ ID NO: 26 - DNA, 26 bp, Artificial
  lntEc154-3′ primer
gtggttacagttcggctattctgcag

SEQ ID NO: 27 - DNA, 24 bp, Artificial
  lntEc190-3′ primer
tggcgttggtcaaacgcactgcag

SEQ ID NO: 28 - DNA, 24 bp, Artificial
  lntEc218-3′ primer
agtggtttaccccacaacctgcag

SEQ ID NO: 29 - DNA, 24 bp, Artificial
  lntEc476-3′ primer
taaccactaacgtgacgcctgcag

SEQ ID NO: 30 - DNA, 29 bp, Artificial
  lntEc513-3′ primer
gagtctgcgtcagcgacgtaaacctgcag

SEQ ID NO: 31 - protein, 7 aa, artificial
  Lnt conserved motif
GXXXCVX

(19) United States
(12) Patent Application Publication
(10) Pub. No.: US 2006/0078952 A1
(43) Pub. Date: Apr. 13, 2006

(54) Method for identifying antimicrobial molecules which interfere with apolipoprotein N-acyltransferase activity

(76) Inventors: Carine Robichon, Paris (FR); Anthony Pugsley, Paris (FR); Dominique Vidal-Ingigliardi, Ermont (FR)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 11/224,952

(22) Filed: Sep. 14, 2005

Related U.S. Application Data
(60) Provisional application No. 60/623,860, filed on Nov. 2, 2004.
(60) Provisional application No. 60/609,241, filed on Sep. 14, 2004.

(51) Int. Cl. G01N 33/554; C07H 21/04; C12P 21/06; C12N 9/10; C12N 1/21; C12N 15/74; G01N 33/569

(52) U.S. Cl. 435/7.32; 435/69.1; 435/193; 435/252.33; 435/488; 536/23.2

(57) ABSTRACT

Products, compositions and methods useful for screening compounds or test molecules for their ability to modulate apolipoprotein N-acyltransferase activity.

### BACKGROUND OF THE INVENTION

1. Field of the Invention

Methods for identifying new anti-microbial compounds using the Lnt protein and cells expressing this protein as targets, especially cells expressing the Lnt protein or a periplasmic segment of Lnt protein under the control of an inducible promoter such as the arabinose/AraC-activated promoter.

2. Description of the Related Art

The apolipoprotein N-acyl transferase (ALP N-acyltransferase) or Lnt protein is known in Escherichia coli. This protein is also historically known as the CutE protein. The Lnt enzyme is responsible for the third step in the processing and fatty acylation of lipoproteins, a class of exported proteins characterized by the presence at their N-terminus of three fatty acids attached to a cysteine residue (Wu, 1996). It has been reported that aminoacylation is essential for the Lol-dependent release of lipoproteins from membranes (Fukuda et al., J. Biol. Chem. 277:43512, 2002). The ALP N-acyltransferase is essential for the growth and viability of S. typhimurium (Gupta et al., J. Biol. Chem. 268:16551, 1993). A temperature-sensitive mutant SE5312 of the Lnt protein (lnt^{ts}) has been identified in Salmonella enterica, sv Typhimurium (Gupta et al., J. Biol. Chem. 268:16551, 1993).

### INTRODUCTION

The Escherichia coli K-12 genome encodes almost a hundred putative lipoproteins (1), a unique class of exported proteins, most of which are anchored in inner leaflet of the outer membrane by their N-terminal fatty acids (2). The best-characterized lipoprotein is Lpp, a trimeric protein (3), that is present in two forms in the outer membrane. Approximately one third of the Lpp molecules in the cell are cross-linked to the peptidoglycan via the C-terminal lysine residue (4), thereby, contributing to outer membrane integrity (5). The remainder of the Lpp exists as a free (unbound) form. Lpp has been extensively used to study bacterial lipoprotein biogenesis and sorting to the outer membrane. Three fatty acids are bound to its N-terminal cysteine residue, two in a diacylglyceride that is linked via a thioether bond to the sulfhydryl group and one to the amine group that is liberated upon signal peptide cleavage (6,7). The fully matured lipoprotein is then detached from the plasma membrane by the ABC transporter LolCDE (8), captured by the periplasmic lipoprotein chaperone LolA (9) and delivered to the outer membrane docking protein LolB (10), whereupon it inserts into the inner lipid leaflet of the outer membrane. It seems reasonable to assume that most if not all outer membrane lipoproteins follow exactly the same route.

A relatively small number of lipoproteins remain in the E. coli plasma membrane. In the two characterized plasma membrane lipoproteins, the endogenous protein NlpA (11) and the Klebsiella oxytoca amylolytic enzyme pullulanase (12), retention in the plasma membrane requires the aspartate (D) residue at position +2, immediately after the fatty acylated cysteine. Furthermore, the introduction of a D+2 residue into outer membrane lipoproteins or its presence in artificial lipoproteins (formed by fusing the signal peptide and first few amino acids of a lipoprotein to a reporter protein) causes their retention in the plasma membrane (13,14). Conversely replacement of D+2 by most other amino acids causes plasma membrane lipoproteins (or artificial lipoproteins) to be routed to the outer membrane (13,14). The ability of D+2 to cause efficient plasma membrane retention is influenced both by amino acids in the adjacent sequence (15,16) and by the structure of the polypeptide of which it is part (17). D+2 lipoproteins differ from outer membrane lipoproteins in being unable to activate the LOlCDE ATPase in proteoliposomes, suggesting that D+2 functions as a Lol-avoidance signal (8,18,19). Other details of the mechanism by which lipoproteins are retained in the plasma membrane are unclear. The unique physico-chemical properties of aspartate are important for this function (20), which makes it all the more surprising that the structurally unrelated aromatic amino acids and proline at position +2 in an artificial lipoprotein can also function as efficient plasma membrane retention signals (14). Furthermore, it has not been established whether D+2 functions as a Lol avoidance signal in other species of bacteria. The plasma membrane enzyme that carries out the third and final step in lipoprotein processing and acylation (apolipoprotein N-acyltransferase or Lnt) was first identified in E. coli (21). The gene (lnt) encoding this enzyme was subsequently identified by the same group through studies of a temperature sensitive Salmonella enterica sv Typhimurium mutant in which apoLpp (lacking the N-acyl group) accumulated at the non-permissive temperature (22). The lnt homologue in E. coli (cutE, referred to here as lnt to reflect its known function) was independently identified in a copper sensitive mutant (23). However, the relationship between Lnt activity and copper sensitivity is unclear. Interestingly, a transposon insertion in the same gene of Rhizobium meliloti (actA) increased copper and acid sensitivity (24). Sequence alignments reveal that homologues of Lnt are present in all Gram-negative bacteria but absent from most Gram-positive bacteria, including Bacillus subtilis and Staphylococcus aureus (25,26). This observation led to the proposal that lipoproteins produced by Gram-positive bacteria might be incompletely fatty acylated and, since all of them are retained in the plasma membrane, that N-acylation might be a characteristic of outer membrane lipoproteins from Gram-negative bacteria (14,25). In apparent agreement with this idea, LolCDE in proteoliposomes cannot promote the capture by LolA of the “apo” form of another major E. coli outer membrane lipoprotein, Pal, suggesting that N-acylation is required for efficient recognition of nascent outer membrane lipoproteins by the Lol system (27). Mass spectrometry demonstrated that plasma membrane-anchored Lpp with a D+2 residue is fully N-acylated, indicating that this retention signal (or Lol avoidance signal) does not operate by preventing N-acylation (27). Furthermore, analysis of E. coli Lpp produced in B. subtilis (28) and of BlaZ beta-lactamase in S. aureus (26) suggested that at least some polypeptides were N-acylated, indicating that these Gram positive bacteria might possess an enzyme with a similar activity to Lnt.

In apparent contrast to the aforementioned retention of apoPal in proteoliposomes in the presence of LolA, studies with S. enterica carrying a ts mutation in lnt revealed that apoLpp produced at the non-permissive temperature (42° C.) was covalently-bound to the peptidoglycan and could not be extracted from the membranes with the detergent sarkosyl, suggesting that it was localized to the outer membrane (22). This interpretation has been called into question (27), since it is known that Lpp that is artificially retained in the plasma membrane by introduction of D+2 is also cross-linked to the peptidoglycan, leading to cell death (29).

To determine the effects of Lnt depletion in E. coli, the present inventors constructed a conditional lnt mutant in which exclusive expression of the chromosomal lnt gene is tightly regulated from the arabinose-inducible araB promoter. This strategy avoids secondary effects introduced by growth at high temperature necessary to inactivate the thermo-sensitive form of S. enterica Lnt (22). The question of whether apoLpp is retained in the plasma membrane in vivo was then addressed by using a variant of this protein that does not bind to the peptidoglycan (29).

### SUMMARY OF THE INVENTION

Unlike the outer membrane of gram-negative bacteria, the cytoplasmic membrane is relatively impermeable to small molecules. However, many conventional anti-microbial compounds must cross the cytoplasmic membrane into the bacterial cytoplasm before they can exert significant anti-bacterial activity. A limited number of anti-microbial compounds gain access to the cytoplasm by “parasitizing” an uptake system the bacterium uses to absorb nutrients or other molecules into the cytoplasm. Other anti-microbial compounds passively diffuse across the bacterial plasma membrane into the cytoplasm, however this is not always an efficient process. Accordingly, the identification of new anti-microbial compounds which exert anti-microbial activity without having to gain access to the bacterial cytoplasm is of great importance.

It has been found that portions of the Lnt protein are expressed outside the bacterial plasma membrane and thus are accessible to antimicrobial compounds without these compounds having to traverse the bacterial plasma membrane. The Lnt protein is essential maintaining the integrity of the bacterial membrane and thus is a target for antimicrobial agents. Since the Lnt protein appears outside of the bacterial cytoplasm, antimicrobial agents may directly interact with it without having to cross into the cytoplasm.

The search for new antibiotics must start with the identification of new targets. Lnt is such a target. Contrary to current practice, it is inevitable that future antibiotics will be more selective in their spectrums of action. Therefore, targets that are essential in some bacteria, but not in others, must be considered a high-priority. The present inventors have identified the Lnt protein as one such target due to its importance in bacteria, such as Escherichia coli and Salmonella.

The target enzyme is apolipoprotein N-acyl transferase (Lnt), a protein present in E. coli. The inventors genomic analysis has shown that the Lnt protein is present in all other gram-negative bacteria in Mycobacterium, Corynebacterium, Streptomycetes and Deinococcus but not in most of gram-positive bacteria, Archaea or eukaryotes. This enzyme is responsible for the third step in the processing and fatty acylation of lipoproteins, a class of exported proteins characterized by the presence at their N-terminus of three fatty acids attached to a cysteine residue (Wu, 1996). The successive steps in processing are the addition of a diacyl glyceride by lipoprotein signal peptidase (LspA) and N-acylation (FIG. 1)(Wu, 1996). All lipoproteins in gram-negative bacteria are believed to undergo this modification process, but very few lipoproteins have been tested. The antibiotic globomycin is a non-competitive inhibitor of lipoprotein signal peptidase.

Fully acylated lipoproteins are either directed to the outer membrane via the so-called Lol system, or are retained in the plasma membrane. The Lol machinery comprises a three-component plasma membrane ABC transporter (LolCDE)(Yakushi et al, 2000) whose ATPase activity is stimulated upon contact with outer membrane lipoproteins (Narita et al., 2003), a periplasmic chaperon (LolA that captures lipoproteins that are expulsed from the plasma membrane by the ABC transporter (Matsuyama et al., 1995) and LolB, an outer membrane docking protein for lipoprotein-LolA complexes (Matsuyama et al, 1997). The canonical signal that prevents lipoproteins from interacting productively with the Lol machinery is an aspartate residue immediately after the fatty acylated cysteine residue at the lipoprotein N-terminus (Yamaguchi et al., 1988). Other amino acids at this position (tryptophane, phenylalanine, tyrosine and praline) have the same effect (Seydel et al, 1999) but in no case was it clear how they function. In an in vitro system, apolipoprotein (lacking the third fatty acid) is not released from the plasma membrane by LolCDE and, therefore, does not interact with LolA (Fukuda et al., 2002).

The present inventors have found that the Lnt protein is intimately involved in the correct sorting of Lpp to the outer membrane by constructing a bacterial strain in which the Lnt gene is exclusively under the control of an inducible promoter. Moreover, the present inventors have used a combination of various methodologies to map the topology of the Lnt protein and have identified segments of the Lnt protein located on the periplasmic side of the inner membrane as being associated with the Lnt protein activity.

This work has established that the Lnt protein is an attractive target molecule for identifying antimicrobial agents or compounds which can selectively affect gram-negative bacteria. The identification of compounds exerting a selective activity on gram-negative bacteria via interferences with the Lnt protein activity is an important step if identifying antimicrobial agents or compounds with little or no toxicity for mammals and which exert reduced effects on the normal microflora in treated subjects.

With the above in mind, several aspects of the invention present themselves:

An aspect of the invention is a conditional mutant of a gram-negative bacterium, such as Escherichia coli, in which the Lnt protein coding sequence or a sequence encoding a periplasmic segment of Lnt is placed under exclusive control of an inducible promoter, such as the arabinose/AraC-activated promoter. The Lnt protein is expressed when the promoter sequence is activated by the presence of arabinose. Removal of arabinose attenuates transcription of the lnt gene and expression of the Lnt protein. Inducible promoters are known in the art and include for example, the arabinose AraC-activated promoter. Inducible promoters are also described by Current Protocols in Molecular Biology (1987-2004), which is incorporated by reference, see e.g., Chapter 2. Other suitable promoters include rhamnose inducible promoter of PrhaB, nitrite inducible promoter of nirB, cold inducible promoter of cspA, hscA and hscB.

A related aspect is the identification of extra-cytoplasmic domains or segments of the Lnt protein and the use of these domains to identify anti-microbial compounds. For example, it has been found that the temperature-sensitive E435K mutant appears in the last predicted periplasmic loop of the Lnt protein. These extra-cytoplasmic domains are important targets for testing antimicrobial agents. These domains present attractive targets for extracytoplasmic binding of putative antimicrobial agents.

The following sequences correspond to portions of the Lnt protein in the periplasmic space in E. coli:

Residues 28-33 of SEQ ID NO: 2: fspydv (SEQ ID NO: 3)

Residues 76-87 of SEQ ID NO: 2: yvsiatfggmpg (SEQ ID NO: 4)

Residues 212-488 of SEQ ID NO: 2: qwftpqpek tiqvsmvqgd ipqslkwdeg qllntlkiyy nataplmgks sliiwpesai tdleinqqpf lkaldgelrd kgsslvtgiv darlnkqnry dtyntiitlg kgapysyesa drynknhlvp fgefvplesi lrplapffdl pmssfsrgpy iqpplsangi eltaaicyei ilgeqvrdnf rpdtdyllti sndawfgksi gpwqhfqmar mralelarpl lrstnngita vigpqgeiqa mipqftrevl ttnvtpttgl tpyartgn (SEQ ID NO: 5).

The highly conserved (red) and structurally conserved residues (blue) of bacterial Lnt proteins are shown in FIG. 9 and FIG. 10. The residues aligning with the E. coli Lnt periplasmic domains are indicated in FIG. 9 and FIG. 10. The structurally conserved periplasmic residues of Lnt proteins of other bacteria are also aspects of the present invention. The red and/or blue residues identified in FIG. 9 and FIG. 10 and subsequences thereof, especially in the periplasmic domains, form discrete motifs from which useful Lnt target polypeptides may be engineered or identified. For example, polypeptides comprising these motifs may be used in methods for screening new antibiotics active against bacteria from which they are derived.

Compounds identified using the methods described above which affect the activity of the Lnt protein also are aspects of the present invention. Such compounds include chemical compounds, single-chain antibodies and competitive or non-competitive peptides. Such compounds may bind to periplasmic segments of Lnt and prevent their association with each other or with other cellular components, thus blocking or inhibiting Lnt activity.

Such methods may involve complementation of an E. coli lnt mutations, such as a temperature sensitive mutation or of constructs in which the lnt gene is under the control of an inducible promoter, by lnt homologues from other bacteria.

### BRIEF DESCRIPTION OF THE DRAWINGS

The patent or application file contains at least one drawing executed in color. Copies of this patent or patent application publication with color drawing(s) will be provided by the office upon request and payment of the necessary fee.

FIG. 1. Successive steps in lipoprotein maturation in Gram-negative bacteria.

FIG. 2. Construction of the p_{araB}-lnt strain. The kan-rpoCter-p_{araB} cassette was generated by ligation and overlapping PCR amplification of the individual components followed by amplification of the complete cassette (Materials and Methods), which was electroporated into strain BW25113 carrying the plasmid pKD46 (30). Transformants were selected on LB agar containing kanamycin and arabinose. Arrows under the genes indicate the orientation of the transcription units.

FIG. 3. Synthesis and accumulation of apoLpp upon Lnt depletion in E. coli. A. Steady state levels of apoLpp and Lpp. Crude extracts from PAP8504 (p_{araB}-lnt) grown in LB broth containing arabinose or glucose for 8 generations at 37° C. were analyzed by urea SDS-PAGE and by immunoblotting with anti-Lpp antibodies. B. Synthesis of apoLpp. PAP8504 was grown in LB broth containing arabinose or glucose for the indicated number of generations. The cells were then washed and resuspended in minimal medium and labeled for 15 min with 35S-methionine. Lpp was immunoprecipitated and analyzed by urea SDS-PAGE and autoradiography.

FIG. 4. Localization of membrane proteins in E. coli PAP8504. Membranes from PAP8504 (p_{araB}-lnt) grown for 8 generations in LB broth containing arabinose (Lnt+) or glucose (Lnt−) were separated by flotation sucrose density gradient centrifugation. Twenty fractions from each gradient were analyzed by SDS-PAGE and proteins were stained with Coomassie blue (upper panels), or were analyzed by urea SDS-PAGE and immunoblotting with anti-SecG, anti-Pal and anti-Lpp (lower panels). PM, plasma membrane; OM, outer membrane.

FIG. 5. ApoLpp is localized in both membranes in Lnt-depleted cells. Membranes from strain PAP8505 (p_{araB}-lnt, lpp::Tn10) grown in LB medium containing arabinose (Lnt+) or glucose (Lnt−) for 8 generations were separated by flotation sucrose density gradient centrifugation. A. Twenty fractions from each gradient were analyzed by urea SDS-PAGE and immunoblotting with anti-FhuA, anti-SecG, anti-Pal and anti-Lpp. B. Fractions 6-7 and 15-16 corresponding to the plasma and outer membrane fractions from the two gradients were analyzed by urea SDS-PAGE and immunoblotting with anti-Lpp. PM, plasma membrane; OM, outer membrane.

FIG. 6. LipoCA-MalE and NlpD accumulate in the plasma membrane of Lnt-depleted cells. Membranes from strain PAP8505 (p_{araB}-lnt, lpp::Tn10) carrying pCHAP1447 grown in LB medium containing arabinose (Lnt+) or glucose (Lnt−) for 6 generations were separated by flotation sucrose density gradient centrifugation. IPTG was added after 3 generations to induce production of pCHAP1447-encoded lipoCA-MalE with a C-terminal hexahistidine tag, which is under lacZ p control. Fractions from each gradient were analyzed by SDS-PAGE and immunoblotting with anti-FhuA, anti-SecG, anti-Pal, anti-NlpD and anti-His antibodies. PM, plasma membrane; OM, outer membrane.

FIG. 7. Detection by immunoblotting and enzymatic activities of Lnt-PhoA and Lnt-LacZ hybrid proteins in E. coli. A. Crude extracts from KS272 producing Lnt-PhoA and Lnt-LacZ hybrid proteins or carrying plasmids without lnt-derived inserts (control) were analyzed by SDS-PAGE and by immunoblotting with anti-beta-galactosidase and anti-alkaline phosphatase antibodies. B. Alkaline phosphatase and beta-galactosidase activities were measured in permeabilized KS272 producing Lnt-PhoA and Lnt-LacZ hybrids proteins. Enzyme activities (in arbitrary units) are the average of three independent assays. Cells were grown in LB medium containing arabinose to induce expression of the gene fusions.

FIG. 8. Proposed topology of Lnt^{Ec} in the plasma membrane. Arrowheads and numbers indicate the amino acid after which the LacZ and PhoA reporter proteins are fused to Lnt. The position of the E435K substitution in the lnt^{ts} allele of S. enterica is also indicated (diamond).

FIG. 9. Sequence Alignments of Lnt proteins. Total amino acid sequence alignments of Lnt Protein are aligned by bacterial family (alpha protebacteria, beta proteobacteria, gamma proteobacteria, epsilon proteobacteria, delta proteobacteria, Actinobacteria, Cyanobacteria and others). Alignments were performed using version 5.4.1 Multialin (Corpet, F., 1988, Multiple sequence alignment with hierarchical clustering, Nucleic Acids Res. 16:10881-10890). Highly conserved residues are shown in red and structurally conserved residues in blue. “!” indicates I or V, “$” indicates L or M, “%” indicates F or Y and “#” indicates N, D, Q or E. The alignments show the positions of the cytoplasmic (C), transmembrane (TM) and periplasmic (P) domains predicted for the E. coli protein and the regions from periplasmic domain P3 referred to in subsequence alignments D and d. Note that Lnt homologues in epsilon proteobacteria are shorter than in other proteobacteria because they lack a substantial amino-terminal portion of the polypeptide. Alignments of regions D and d show the breadth of conservation of these regions across all proteobacteria (region D) and Actinomycetes, Cyanobacteria, Aquificae, Bacteroides, Deinococcus, Planktomyces, Spirochaetes, and Themotogae also share similarity with E. coli Lnt only in region d. To facilitate observation of this feature, region d is shown for all bacteria. The position of S. enterica SV Typhimurium Lnt residue E435 is shown on all alignments in which it is conserved. Note that R435 is conserved in all proteobacteria except epsilon proteobacteria and Pirullula.

FIG. 10. Additional Sequence Alignments of Lnt proteins. Additional new alignments were obtained as described above for FIG. 9. FIG. 11 mentioned below describes the taxonomy and abbreviations used for various eubacteria.

FIG. 11. Taxonomy and Abbreviations of Eubacteria from which Lnt sequences were derived.

FIG. 12. Immunoblot analysis of recombinant Lnt proteins using monoclonal antibodies directed to the His tag added during the cloning.

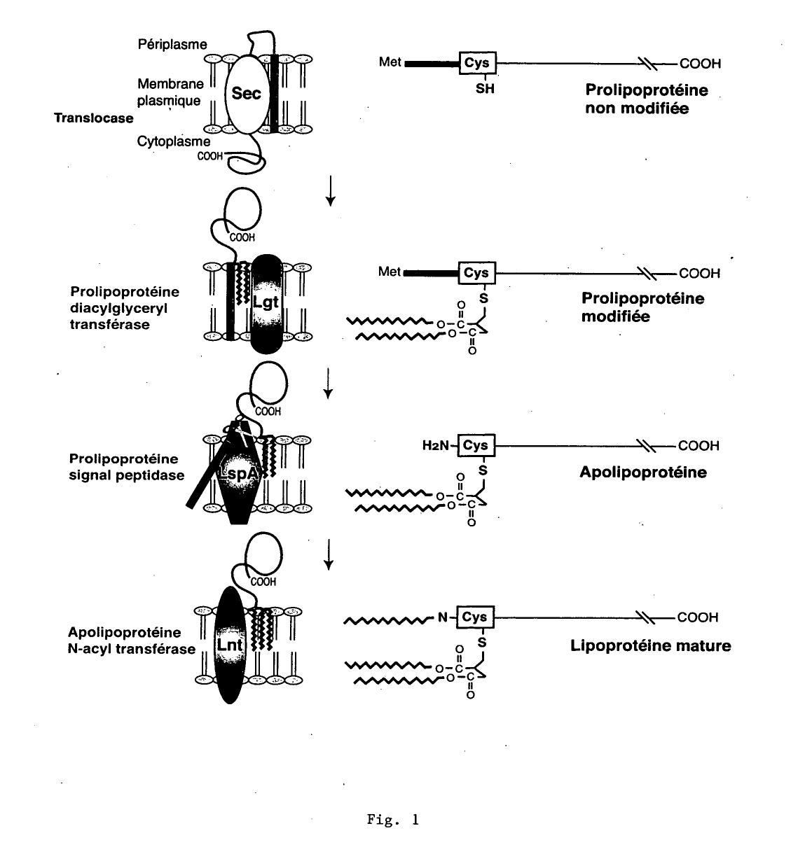

### DETAILED DESCRIPTION OF THE INVENTION

Lipoproteins in Gram-negative bacteria are mainly anchored in the outer membrane, facing the periplasm, through lipids fixed to their N-terminal cysteine. Relatively few lipoproteins remain in the plasma membrane. The two groups of lipoproteins are distinguished by the amino acid at position +2, immediately after the fatty acylated cysteine. It was recently shown that, in vitro, the last step in lipoprotein maturation, N-acylation by lnt gene-encoded apolipoprotein N-acylated transferase (Lnt), is necessary for efficient recognition of outer membrane lipoproteins by the Lol system, which transports them from the plasma- to the outer membrane (Fukuda et al. 2002. J. Biol. Chem. 277:43512-43518). To study the role of Lnt in sorting of lipoproteins in vivo, we constructed an Escherichia coli conditional lnt mutant. The apo form of peptidoglycan-anchored major outer membrane lipoprotein (Lpp) was shown to accumulate when lnt expression was reduced. Two other outer membrane lipoproteins, NlpD and a fatty acylated variant of the normally periplasmic MalE, also accumulated in the plasma membrane when Lnt was depleted. We also found that Lnt is an essential protein in E. coli and that the lethality is caused, in part, by the retention of apoLpp in the plasma membrane. Topology mapping of Lnt with beta-galactosidase and alkaline phosphatase fusions indicated the presence of 6 plasma membrane-spanning segments. A mutation causing thermosensitivity of Lnt in Salmonella enterica sv Typhimurium (Gupta et al. 1993. J. Biol. Chem. 268:16551-16556) was found to result from a single glutamine to lysine substitution at a highly conserved position in the last predicted periplasmic loop of the protein.

The lnt gene encoding the Lnt (CutE) protein in E. coli is described by Blattner et al., Science 277 (5331), 1453-1474 (1997).

Sequence of the Lnt Gene of E. coli:

(SEQ ID NO: 1)

atggcttttgcctcattaattgaacgccagcgcattcgcctgctgctggc
gttattattcggtgcctgcggaacgctggccttctctccttacgacgtct
ggcctgcggcgattatttcgctgatggggcttcaggcgttgacctttaac
cgccgtccactccagtctgccgctattggcttttgctggggatttggcct
ctttggcagcggtattaactgggtctatgtcagcatcgcgacctttggcg
gaatgcctggcccggttaacatcttcctggtggtgctgctggcggcgtat
ttgtcgctgtataccggactgtttgctggcgtgctgtcgcgtctgtggcc
gaaaaccacctggctgcgcgtagcgattgccgcccctgccctctggcaag
tgaccgagtttctgcgcggttgggtactgaccggcttcccgtggttacag
ttcggctatagccagattgatggcccgttaaaagggctggcaccgataat
gggcgtggaagccattaacttcctgctgatgatggttagtggcctgctgg
cactggcgttggtcaaacgcaactggcgtccgctggtggtggccgtcgtg
ctgtttgcccttcccttcccgctgcgttacatccagtggtttaccccaca
accggagaaaaccattcaggtttcgatggttcagggcgatattccgcaat
cgctgaaatgggacgaaggccagcttcttaatacgctgaagatttactac
aacgcaacggcaccgctgatgggcaaatcatcgttgattatctggccgga
gtcggcgataaccgatctggaaattaatcagcaaccgttcctcaaagcac
tggacggtgagttgcgtgataaaggtagctcgctggtaaccgggattgtc
gacgcgcgtctcaataagcagaaccgctacgatacctacaacaccatcat
cacgctgggtaaaggtgcgccgtacagctacgaatcagccgatcgctata
acaaaaaccatctggtgccgtttggcgagtttgtcccgctggagtcgatt
ctgcgtccgttagcaccgttctttgatctgccgatgtcgtcgttcagccg
tgggccatatatccagccgccgctgtcggcaaatggtattgagcttactg
cggctatttgctacgagatcattctcggcgagcaagtgcgcgataacttc
cgcccggataccgactatctgctgactatctccaacgatgcgtggtttgg
taaatctattggtccatggcaacacttccagatggcgcgaatgcgtgcgc
tggagctggcgcgcccactgttgcgcagcaccaacaacggcattacggcg
gtgattggcccgcagggtgagattcaggcgatgatcccgcagttcacccg
cgaggtgttaaccactaacgtgacgccgaccaccggactcacaccatacg
cacgtaccggcaactggccgctgtgggtgctgacggcattgtttggtttt
gctgctgtgttgatgagtctgcgtcagcgacgtaaataa

Sequence of the Lnt Protein of E. coli:

(SEQ ID NO: 2)

mafaslierqrirlllallfgacgtlafspydvwpaaiislmglqaltfn
rrplqsaaigfcwgfglfgsginwvyvsiatfggmpgpvniflvvllaay
lslytglfagvlsrlwpkttwlrvaiaapalwqvteflrgwvltgfpwlq
fgysqidgplkglapimgveainfllmmvsgllalalvkrnwrplvvavv
lfalpfplryiqwftpqpektiqvsmvqgdipqslkwdegqllntlkiyy
nataplmgkssliiwpesaitdleinqqpflkaldgelrdkgsslvtgiv
darlnkqnrydtyntiitlgkgapysyesadrynknhlvpfgefvplesi
lrplapffdlpmssfsrgpyiqpplsangieltaaicyeiilgeqvrdnf
rpdtdylltisndawfgksigpwqhtqmarmralelarpllrstnngita
vigpqgeiqamipqftrevlttnvtpttgltpyartgnwplwvltalfgf
aavlmslrqrrk.

For the purposes of this invention this gene will also include genes encoded by sequences which hybridize to SEQ ID NO: 1 or its complement under stringent conditions and which encode a polypeptide having N-acyltransferase activity, especially those isolated from gram-negative bacteria which have N-acyltransferase activity.

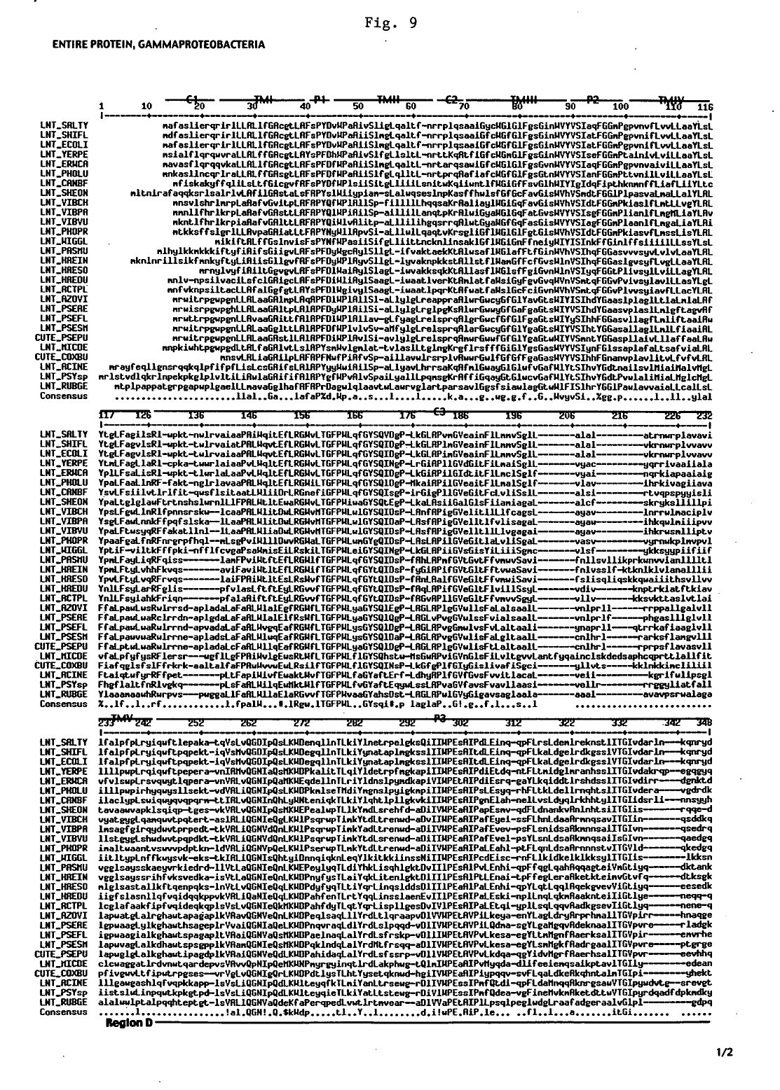

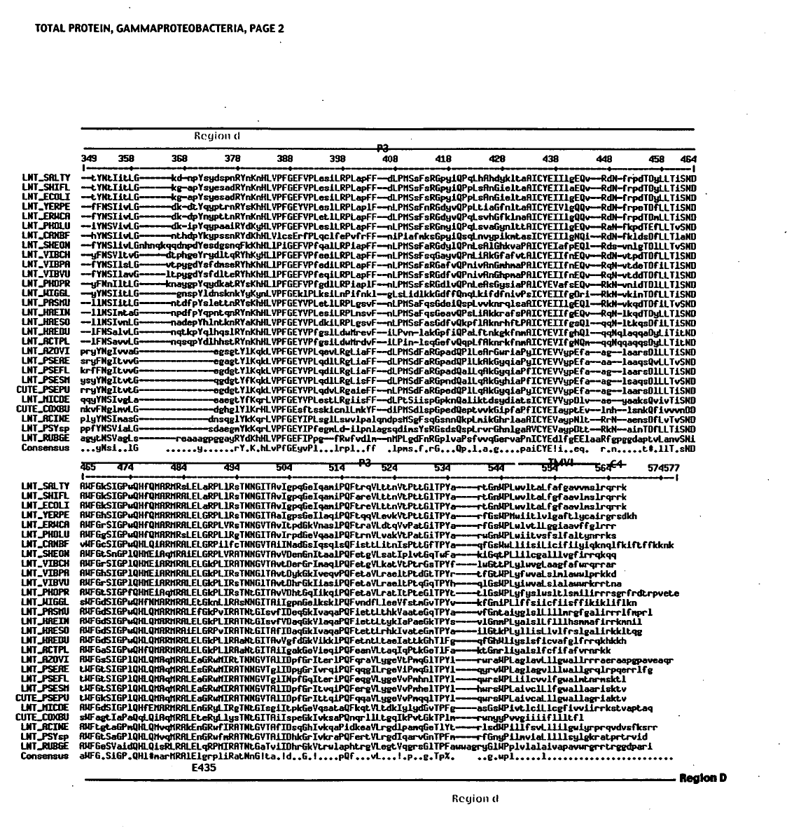

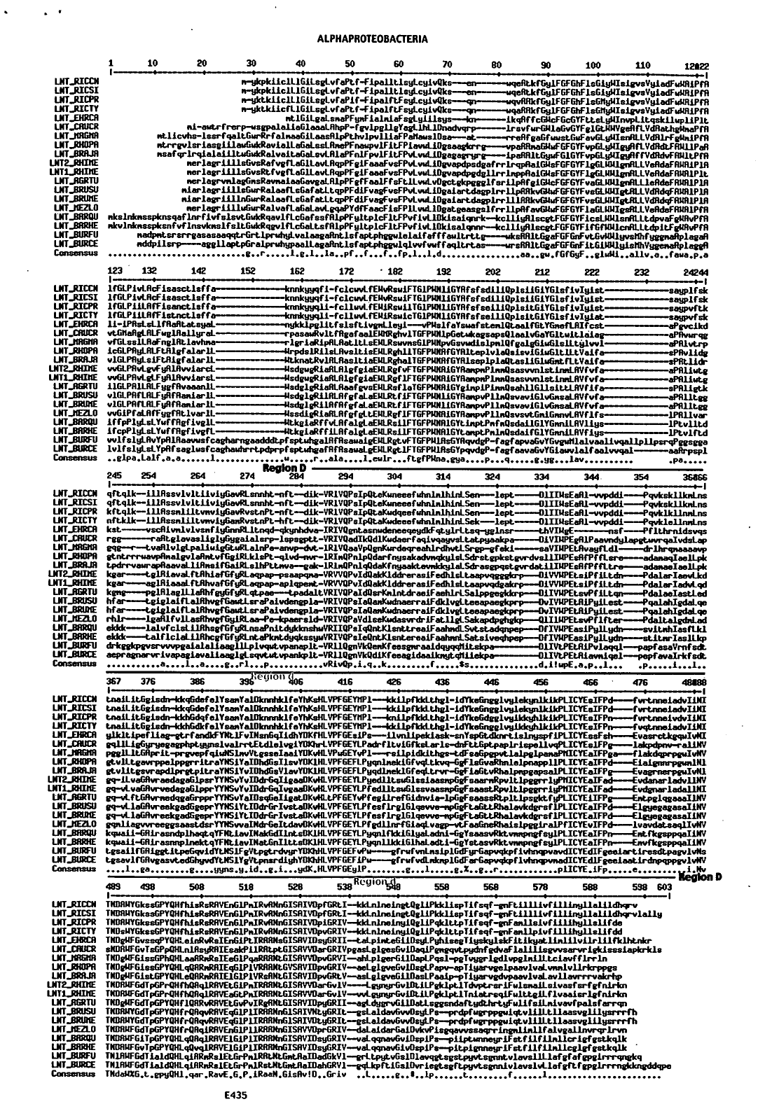

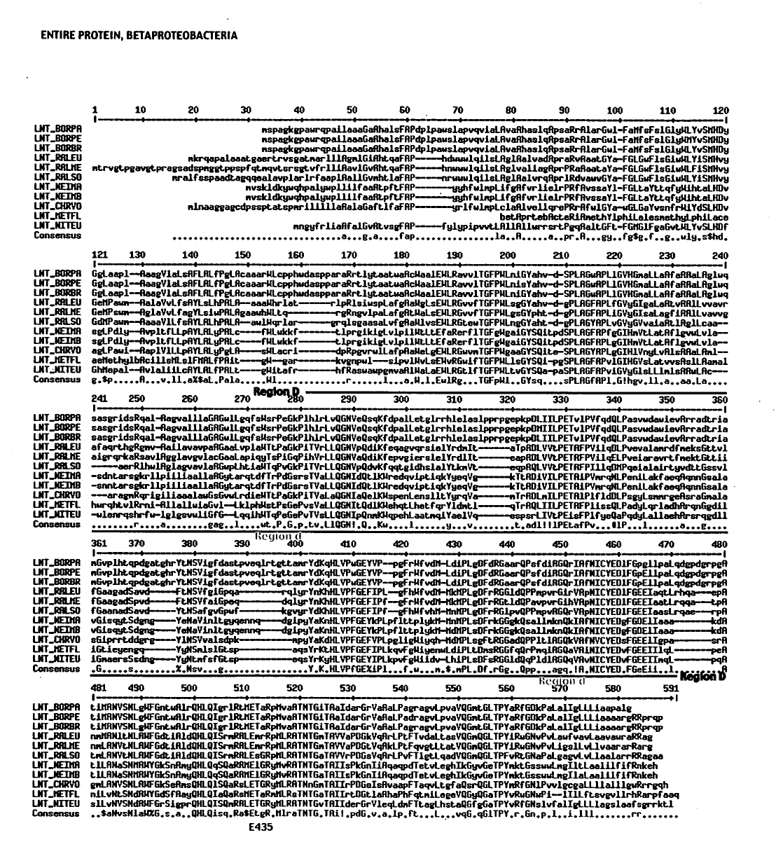

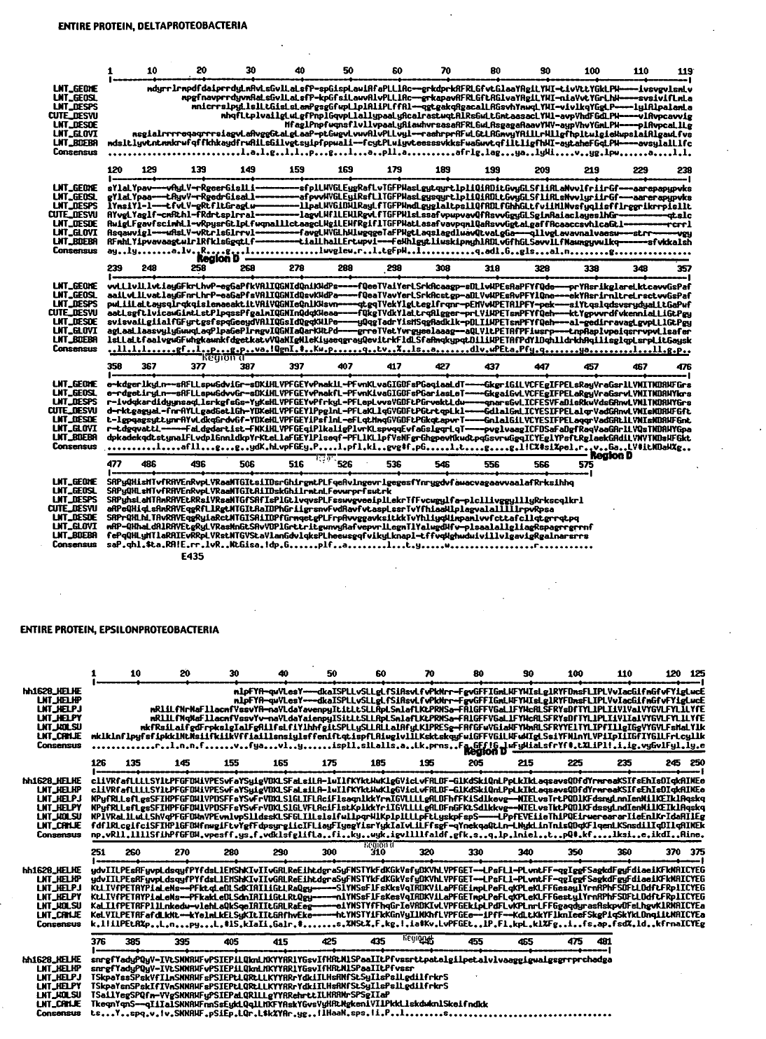

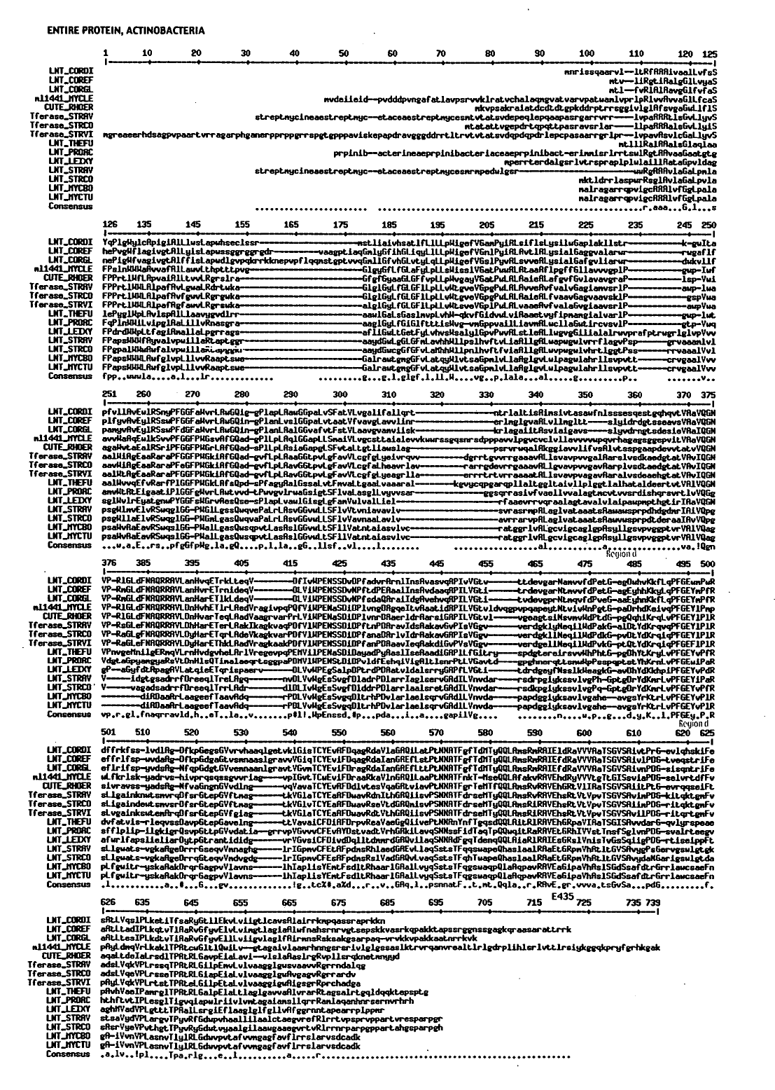

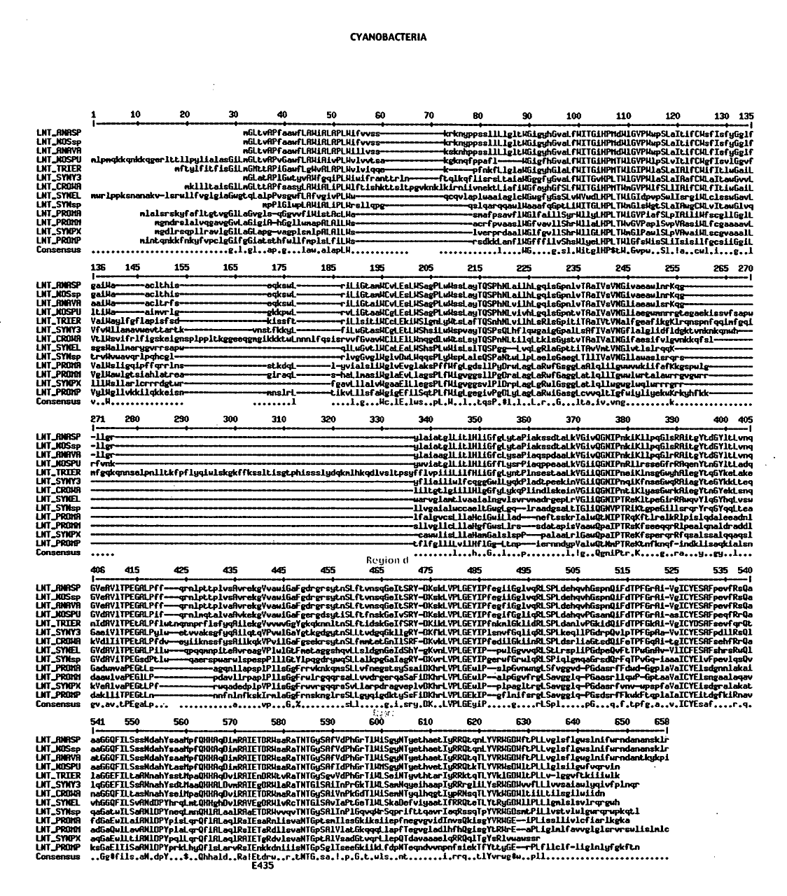

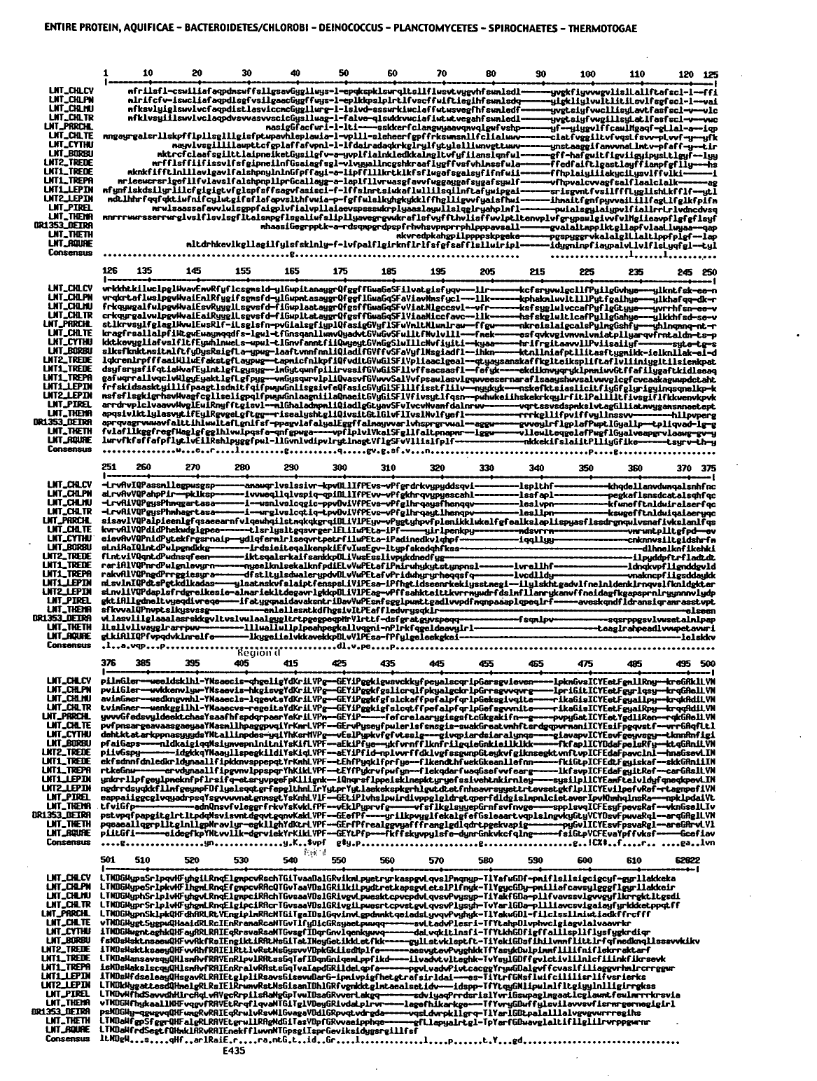

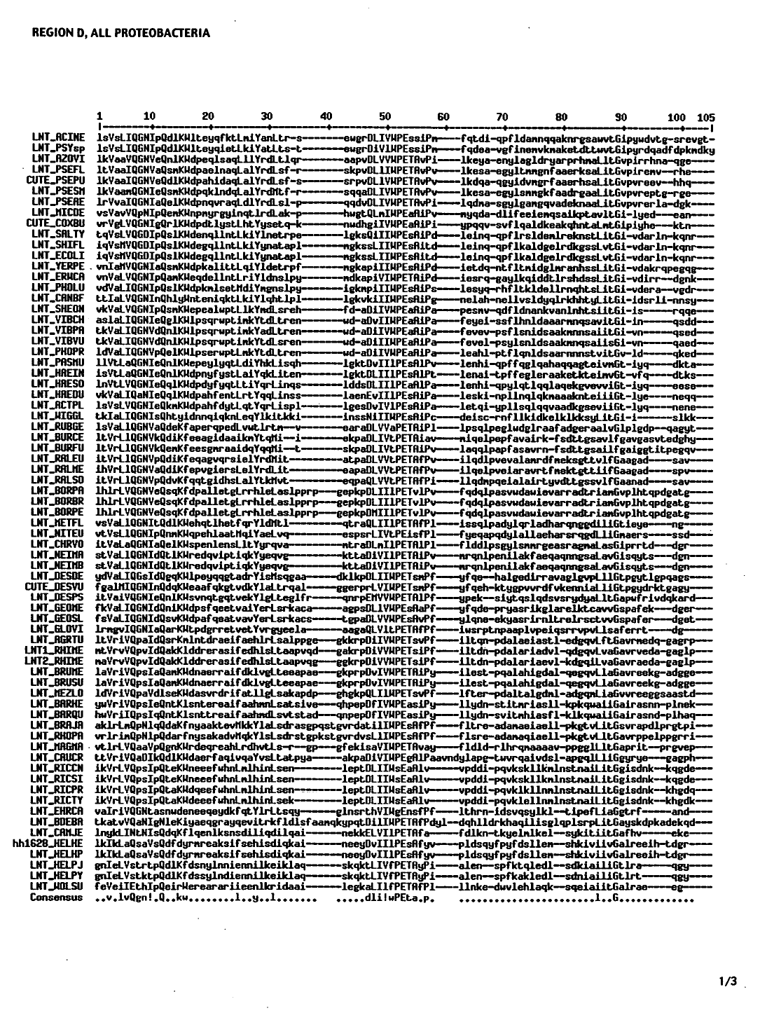

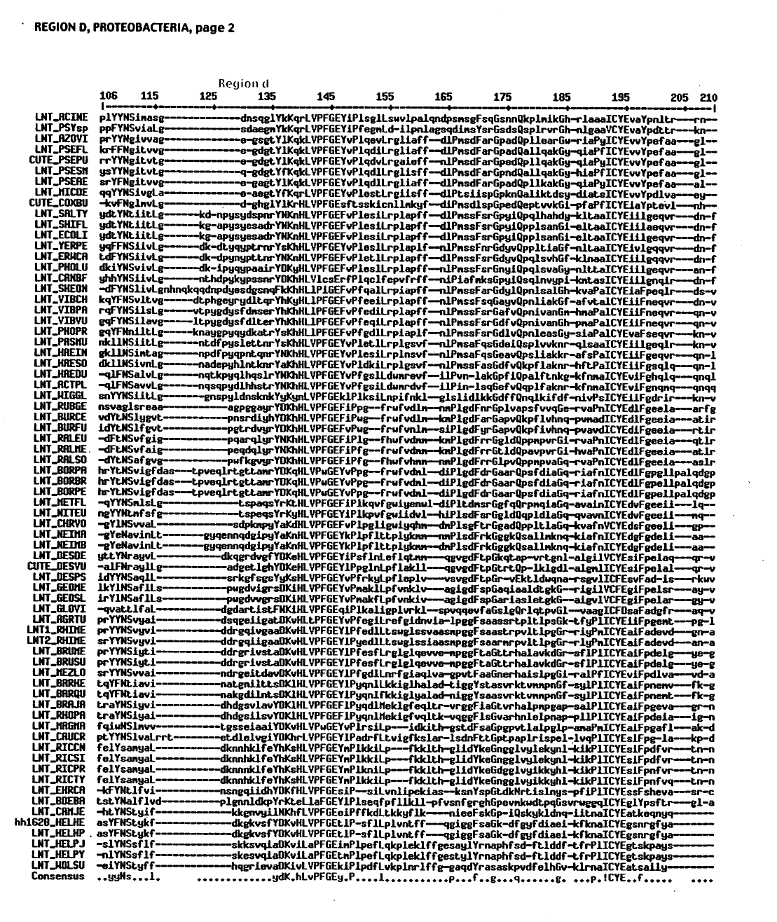

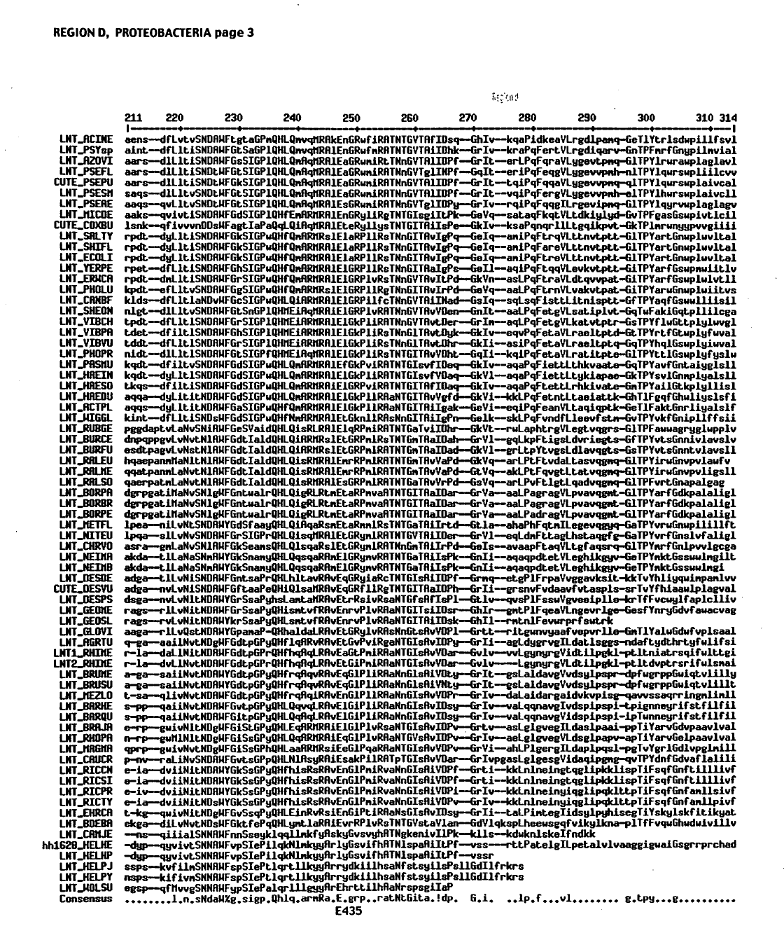

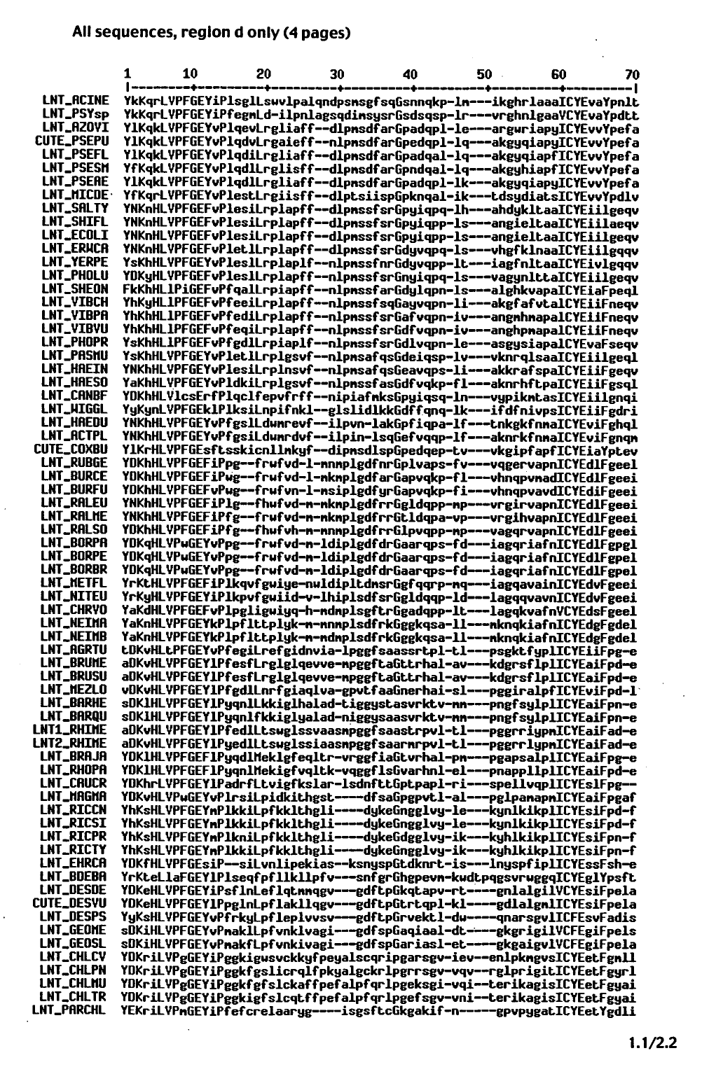

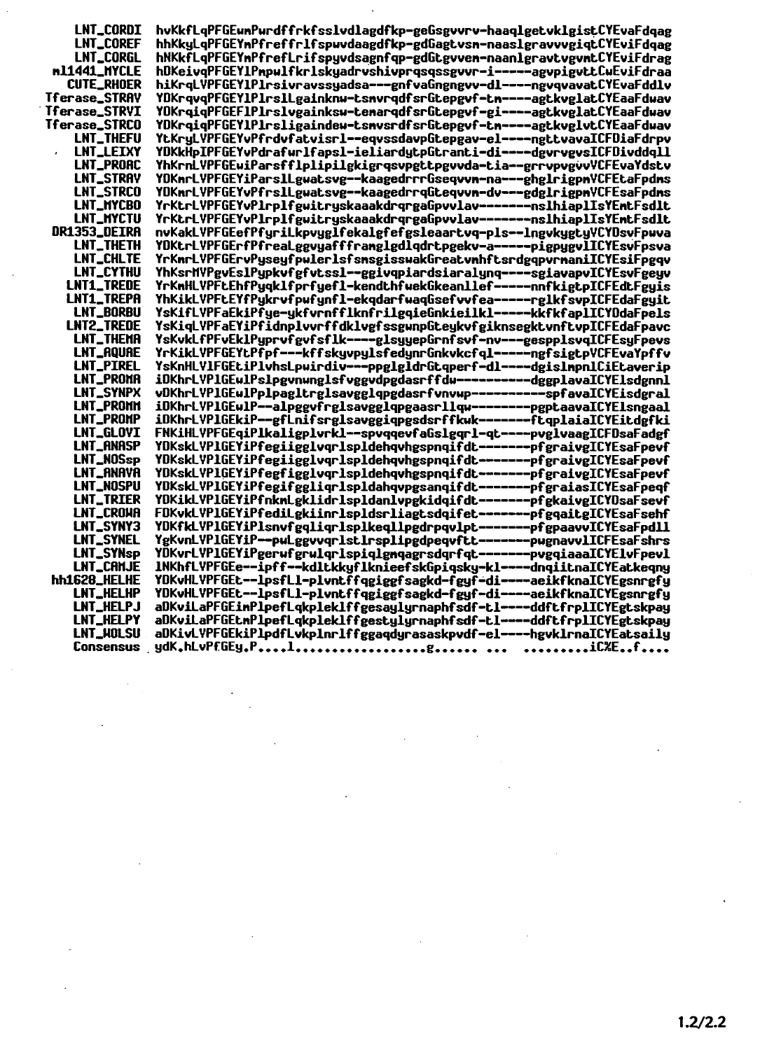

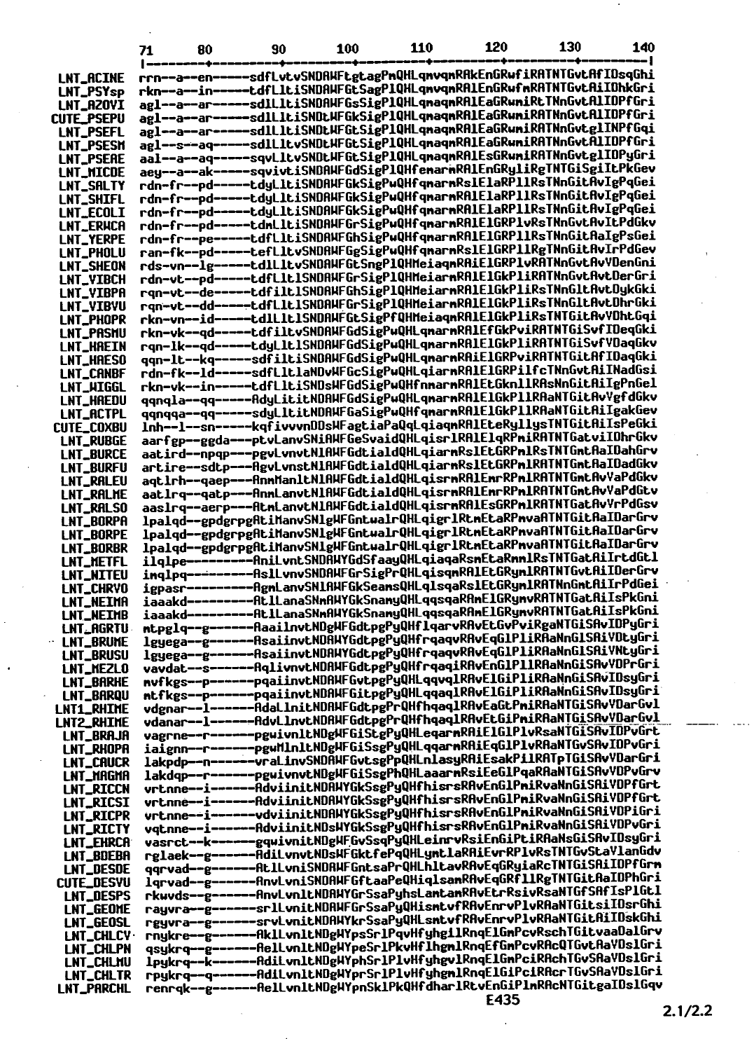

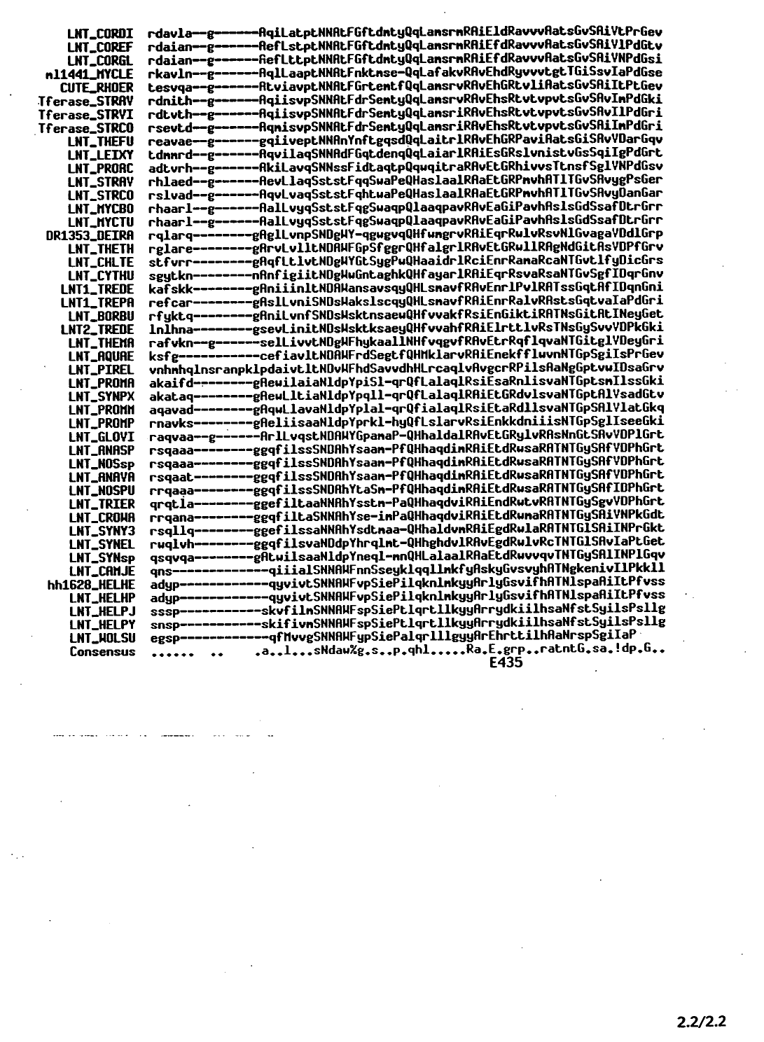

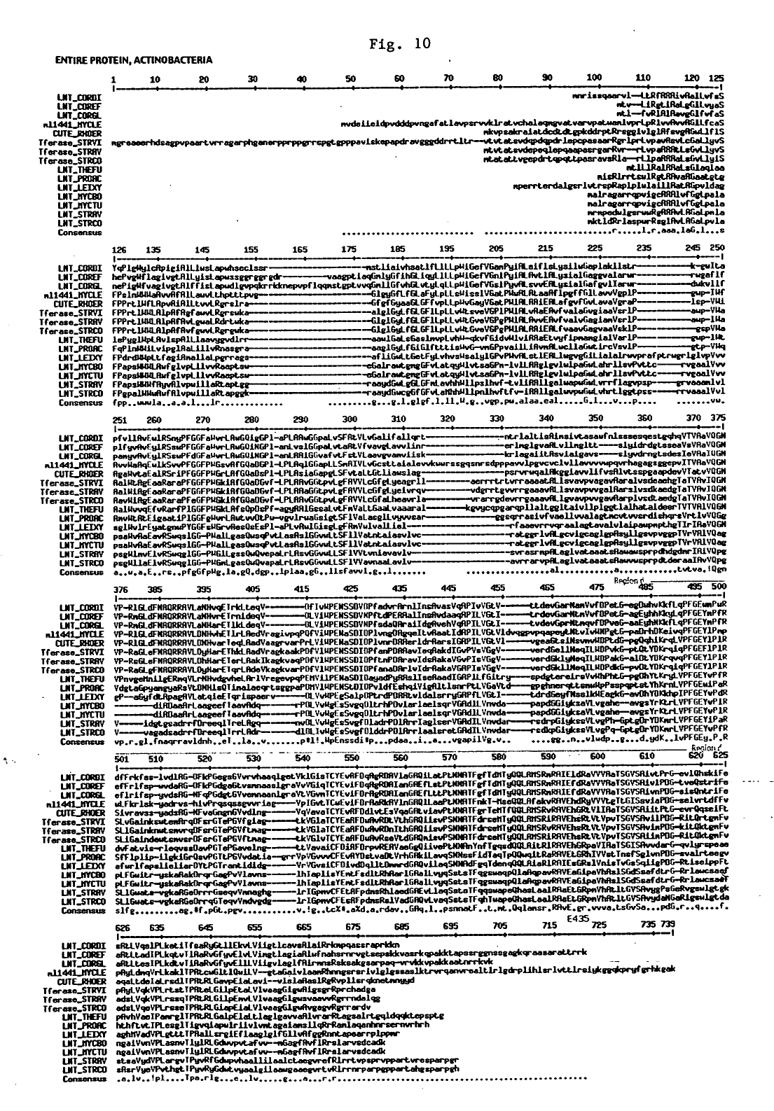

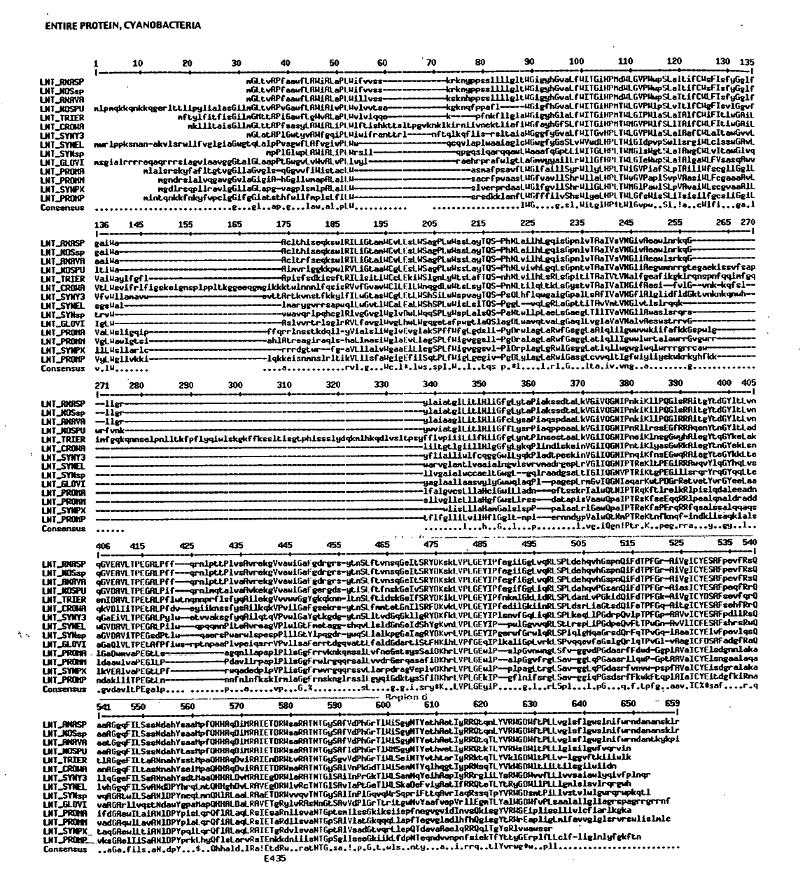

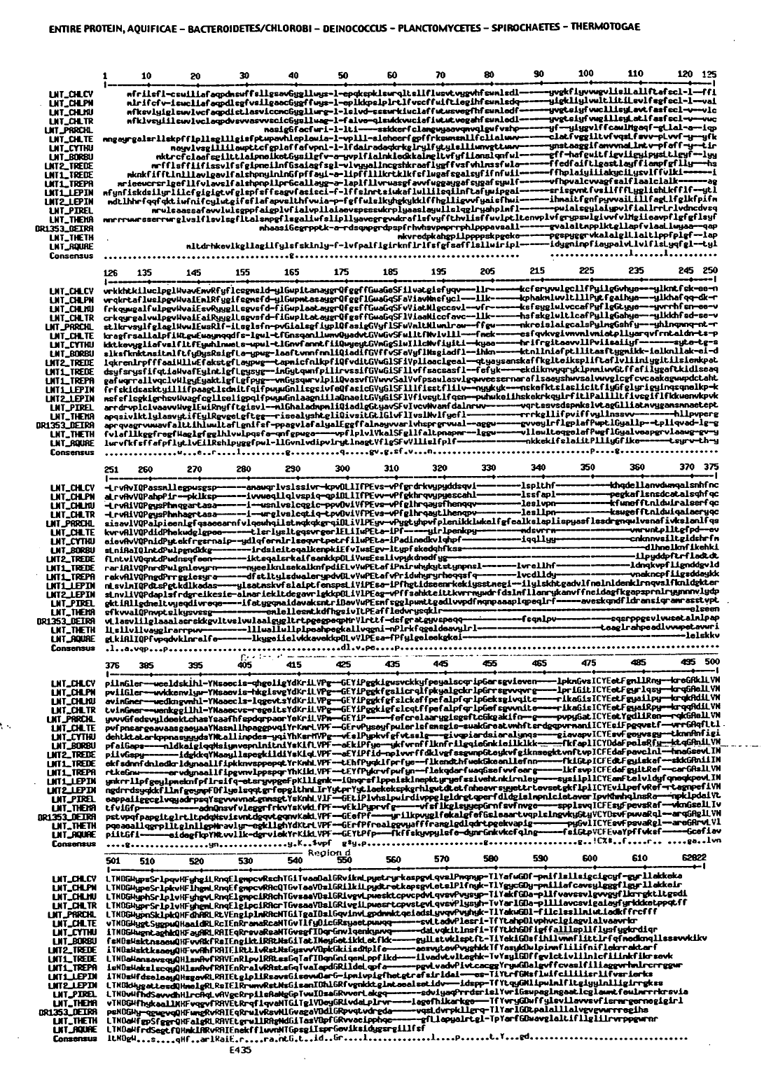

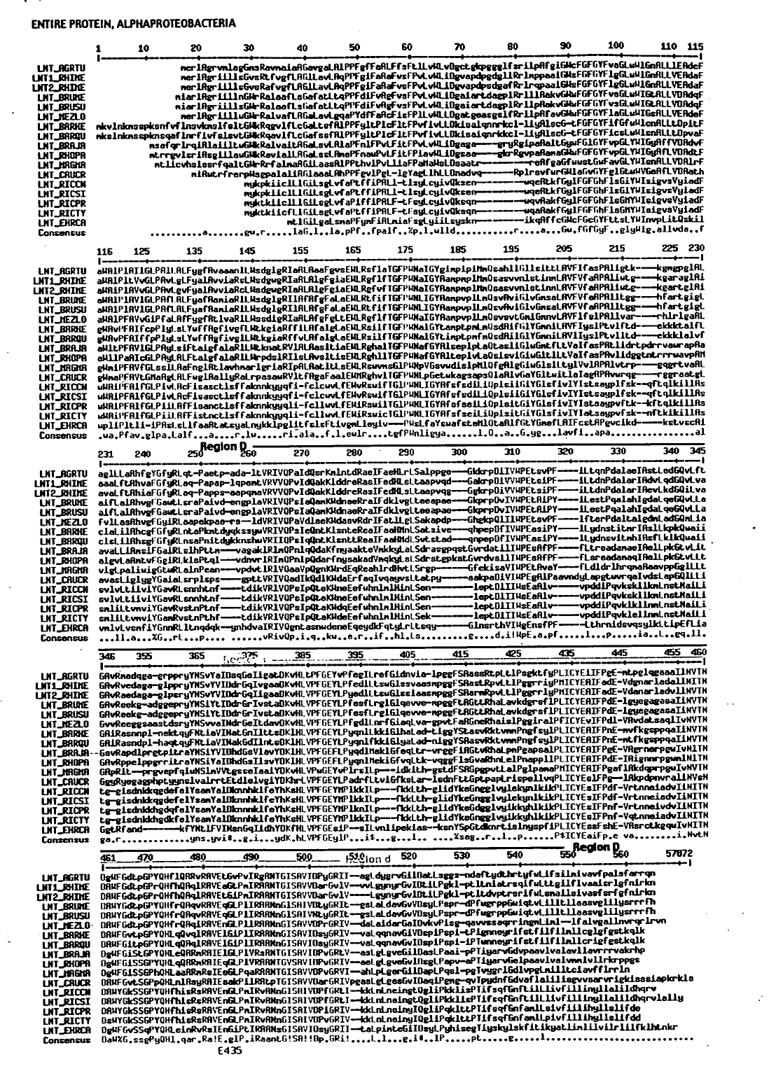

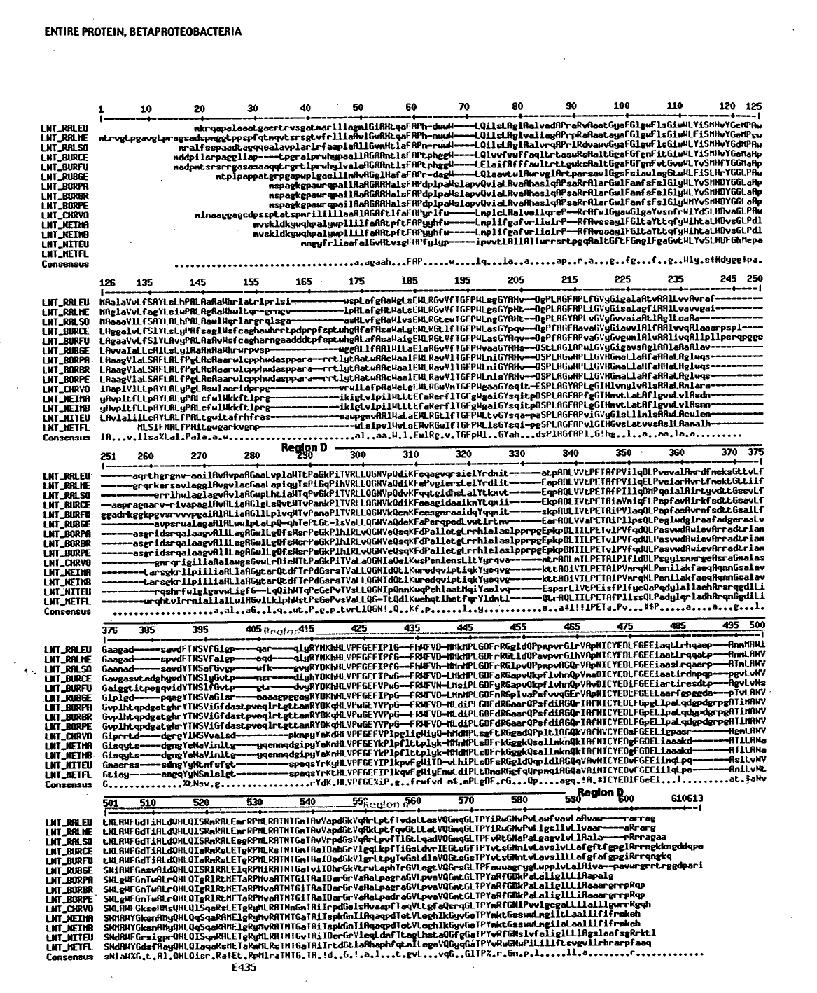

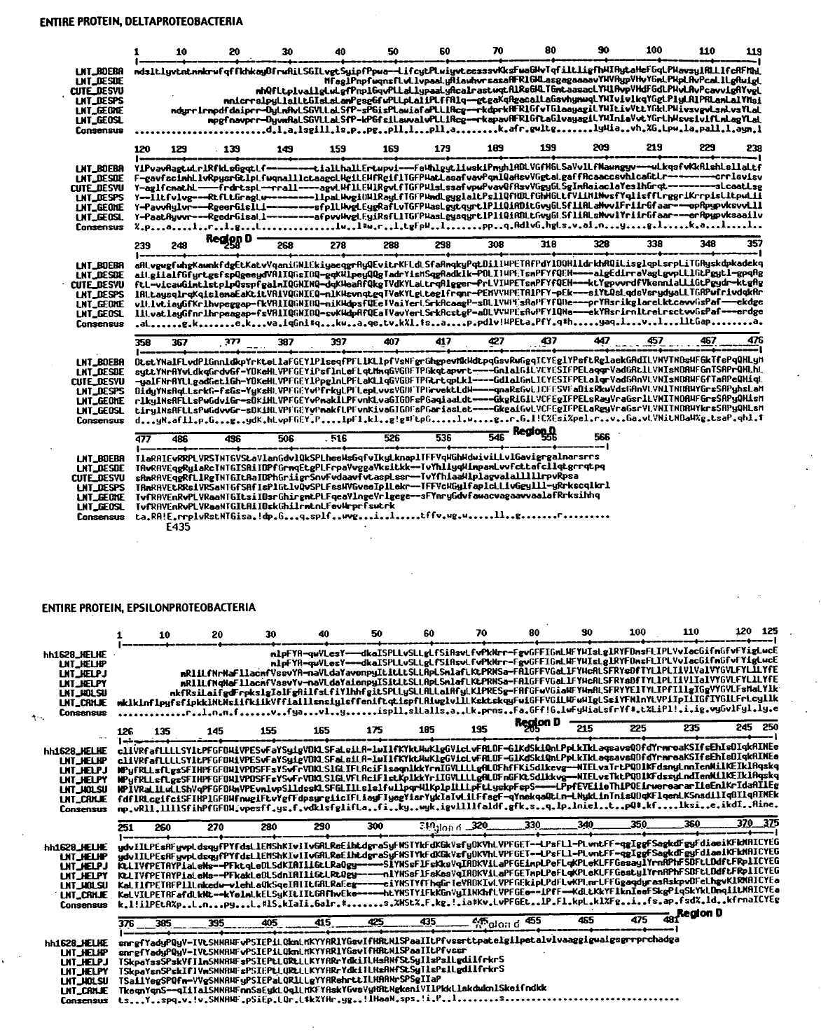

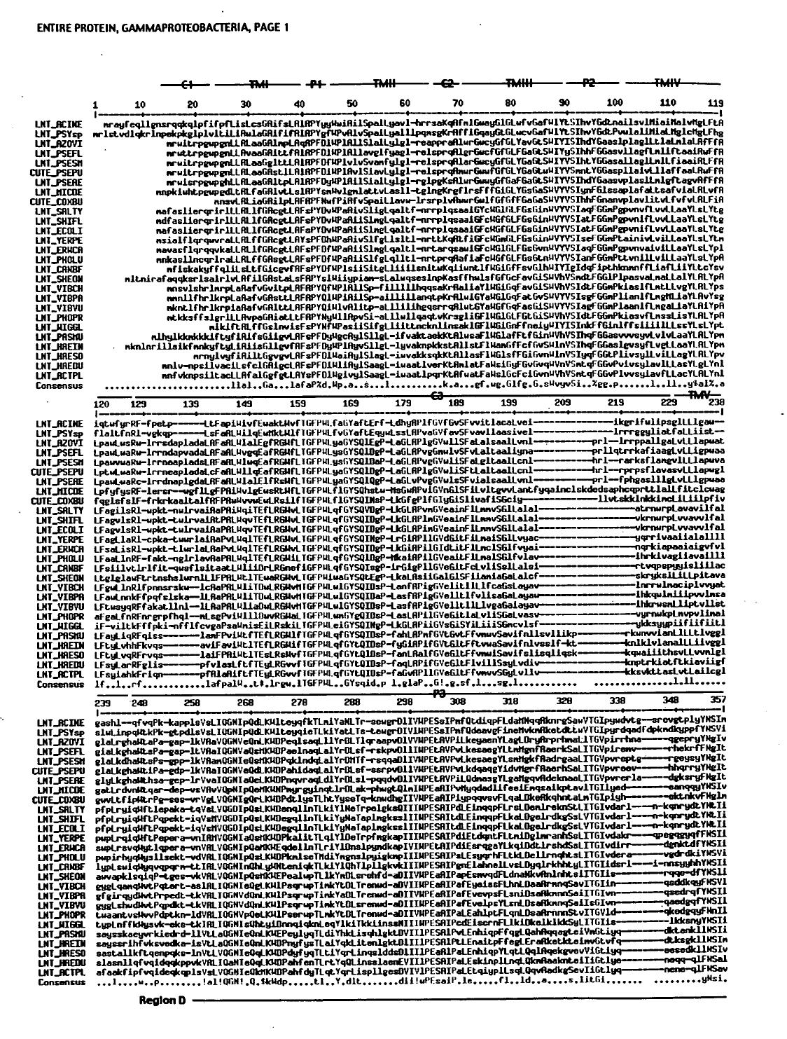

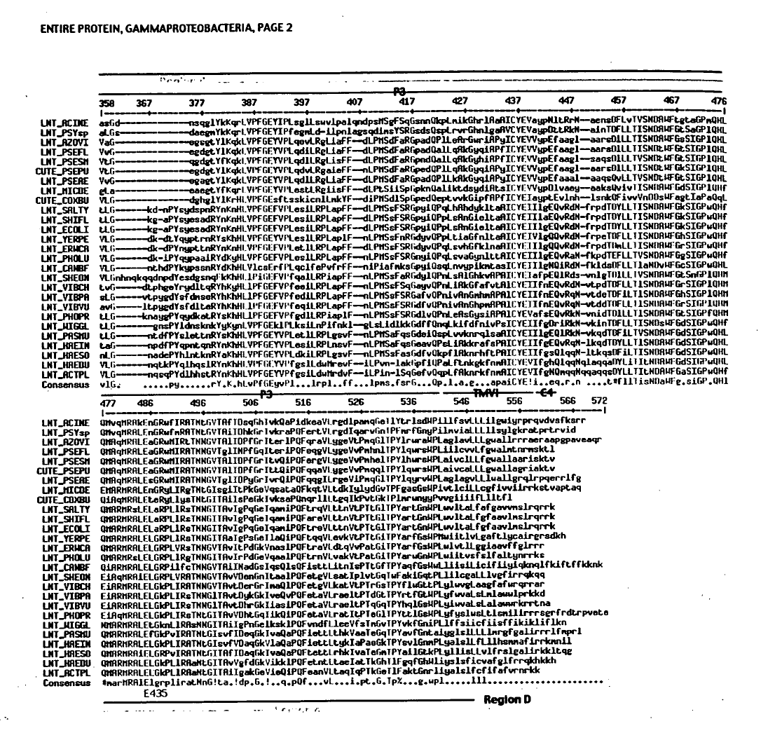

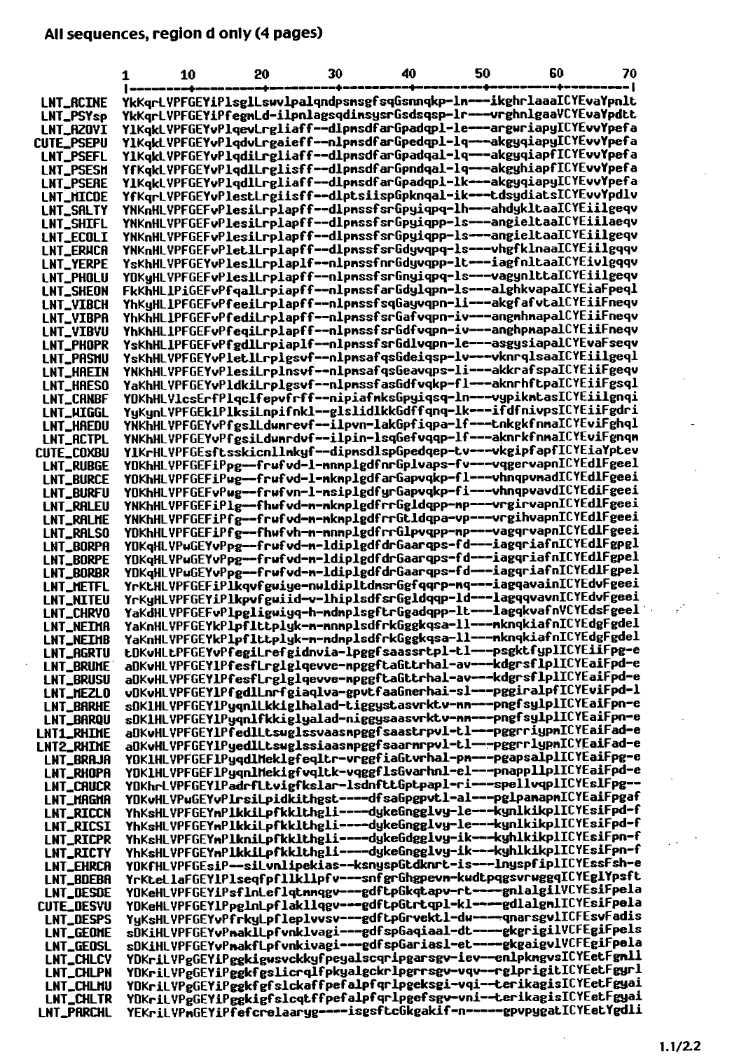

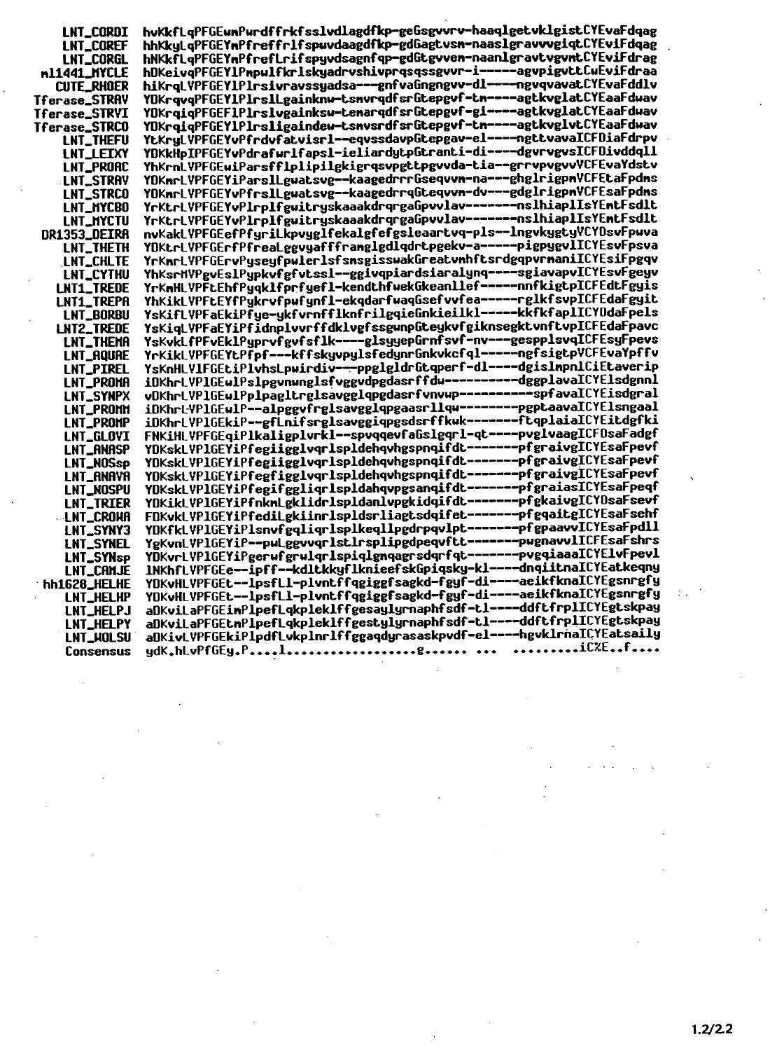

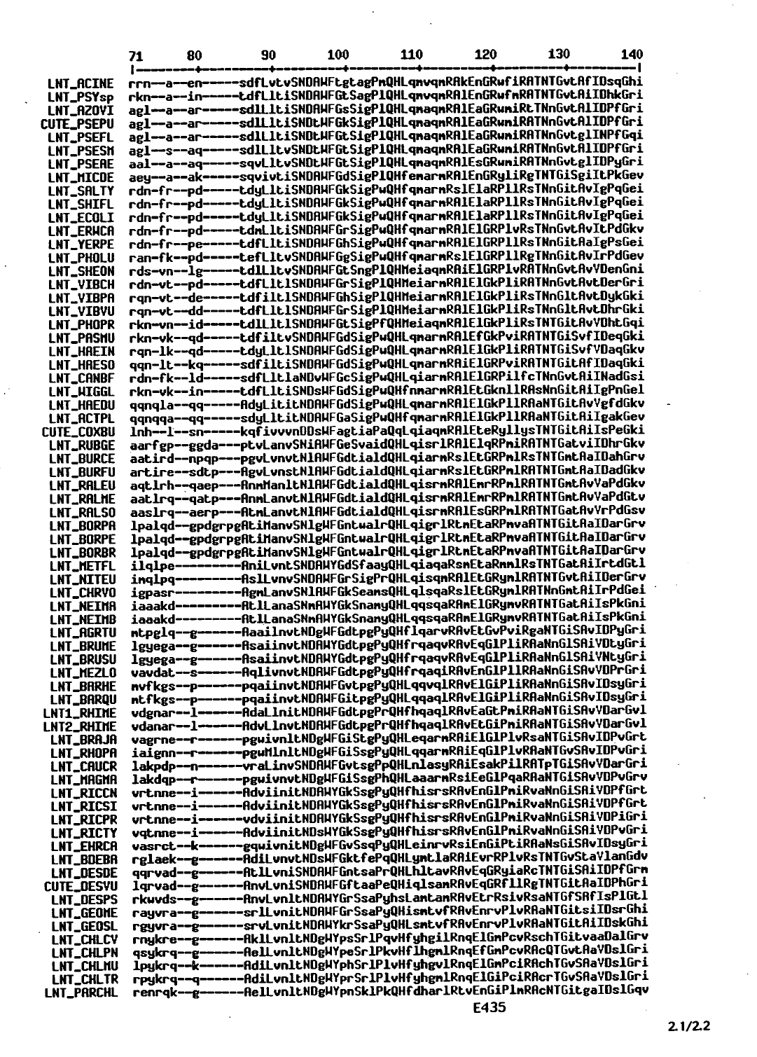

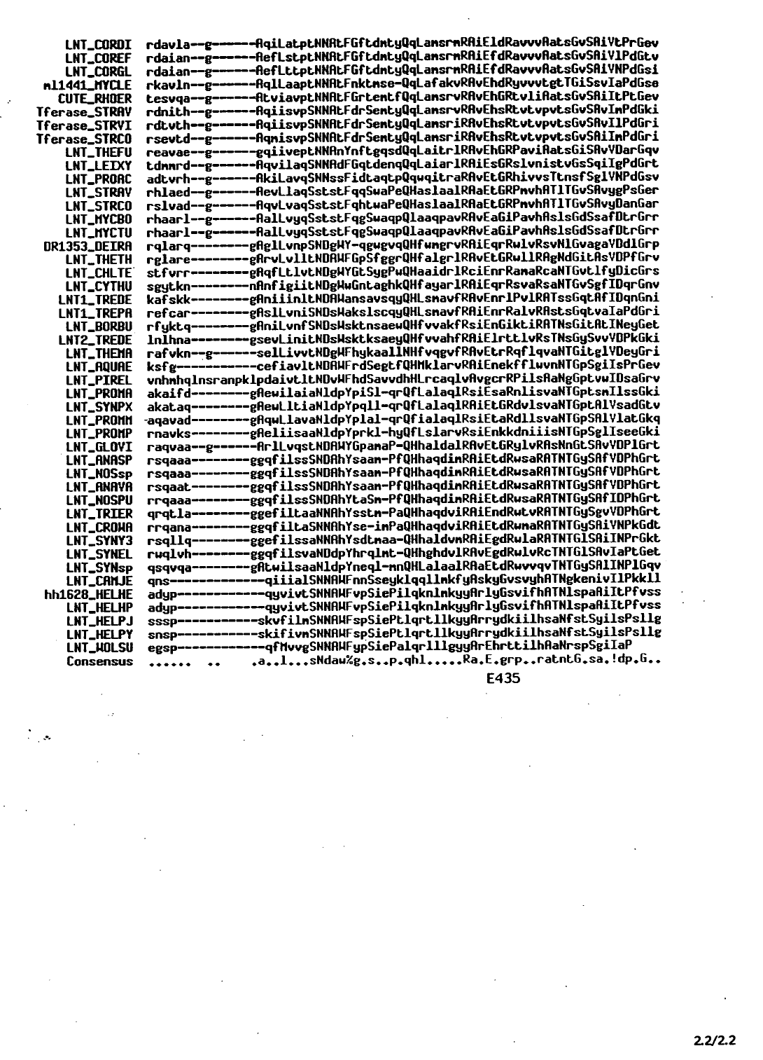

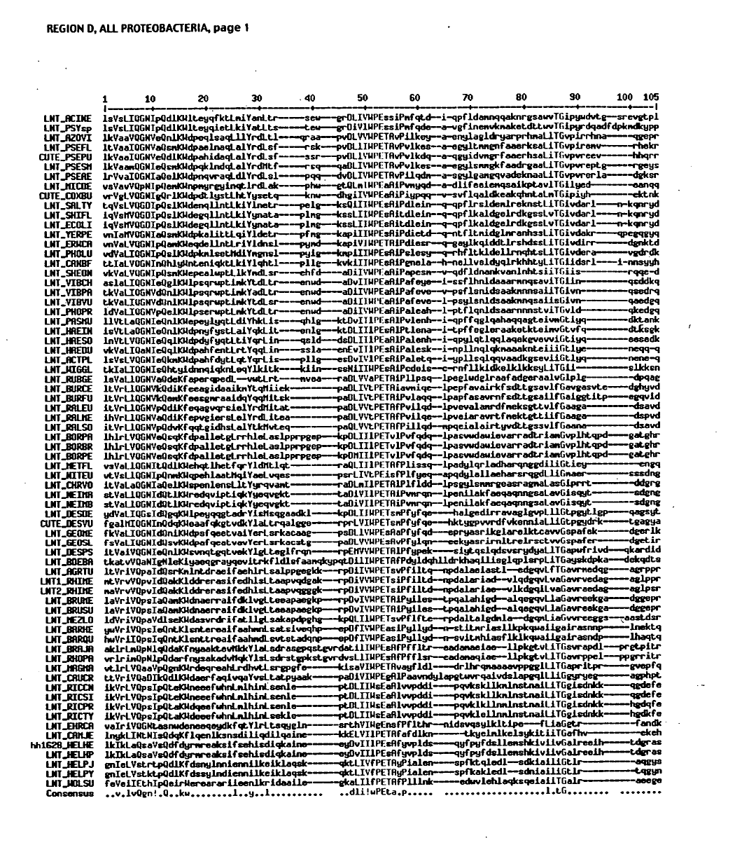

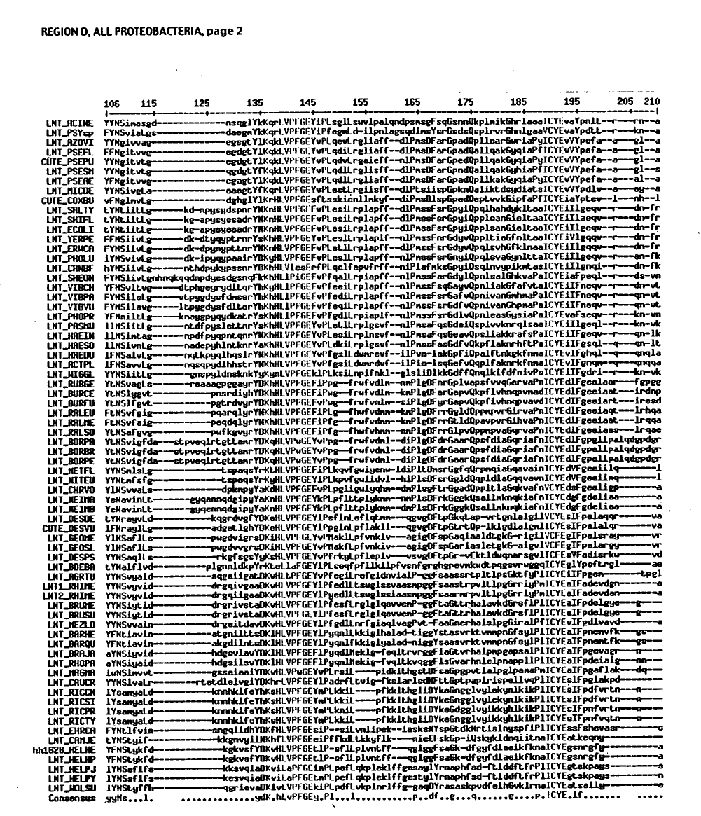

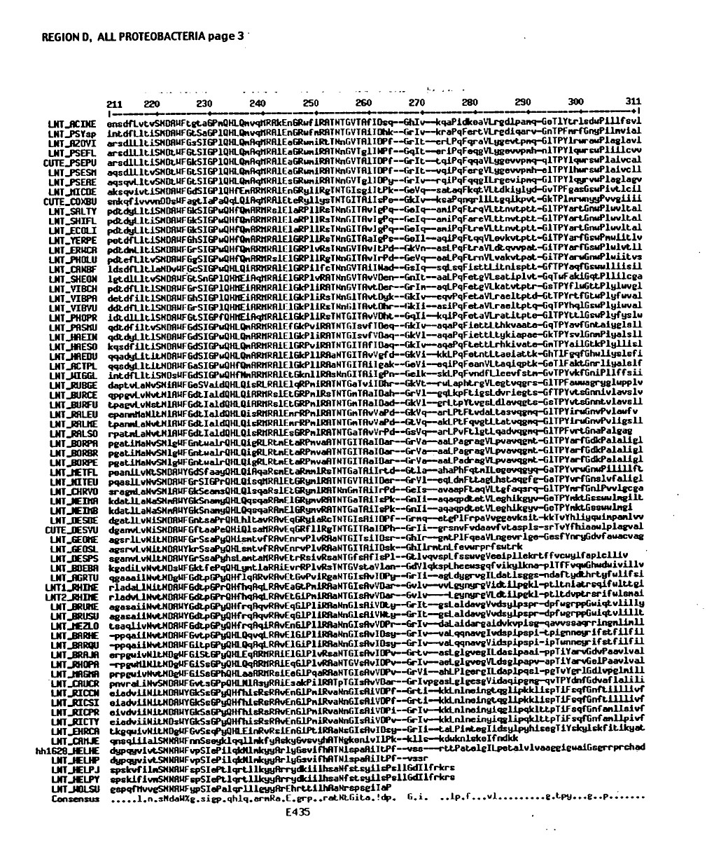

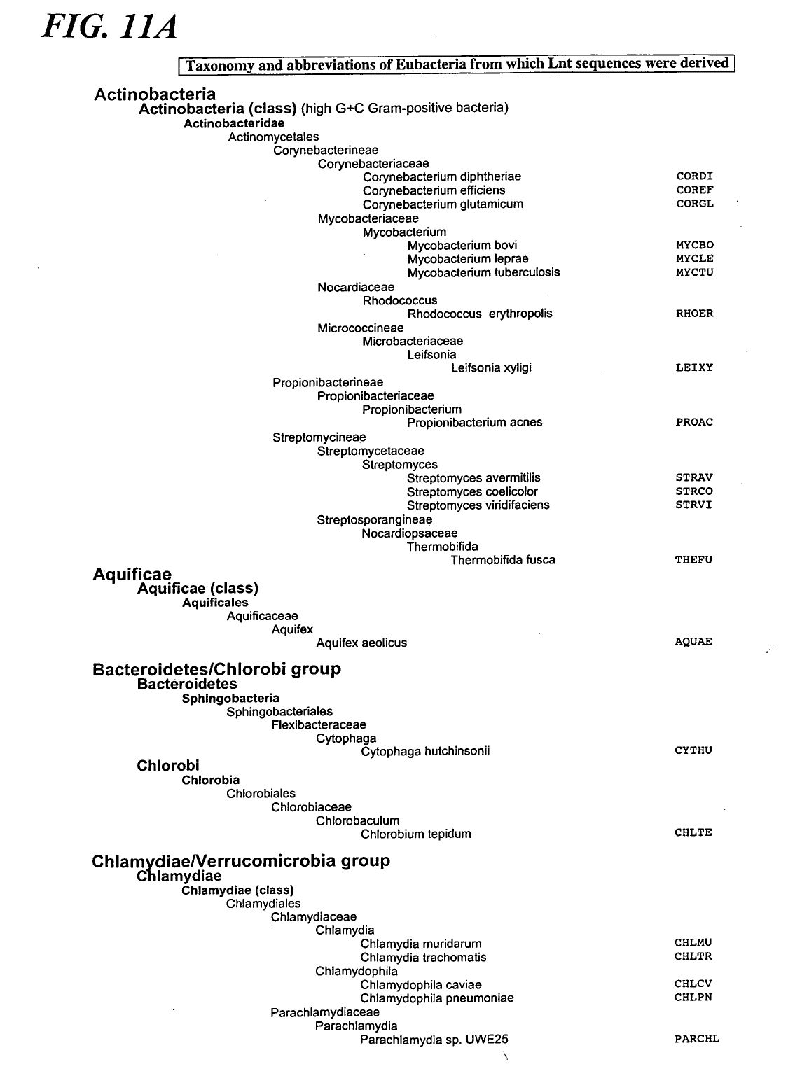

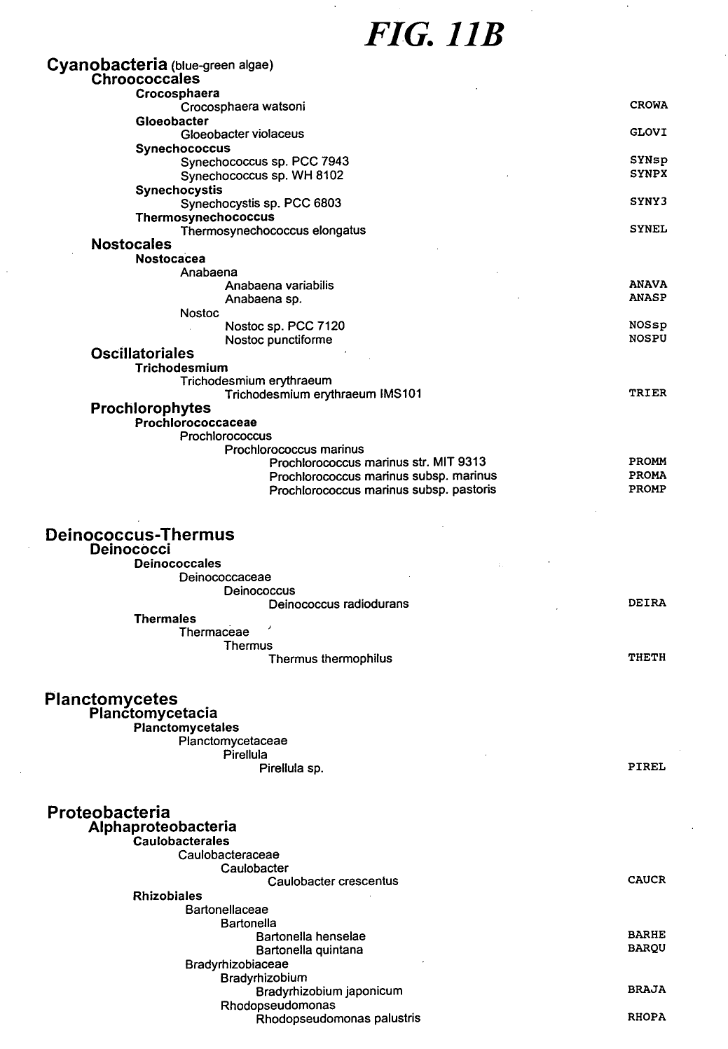

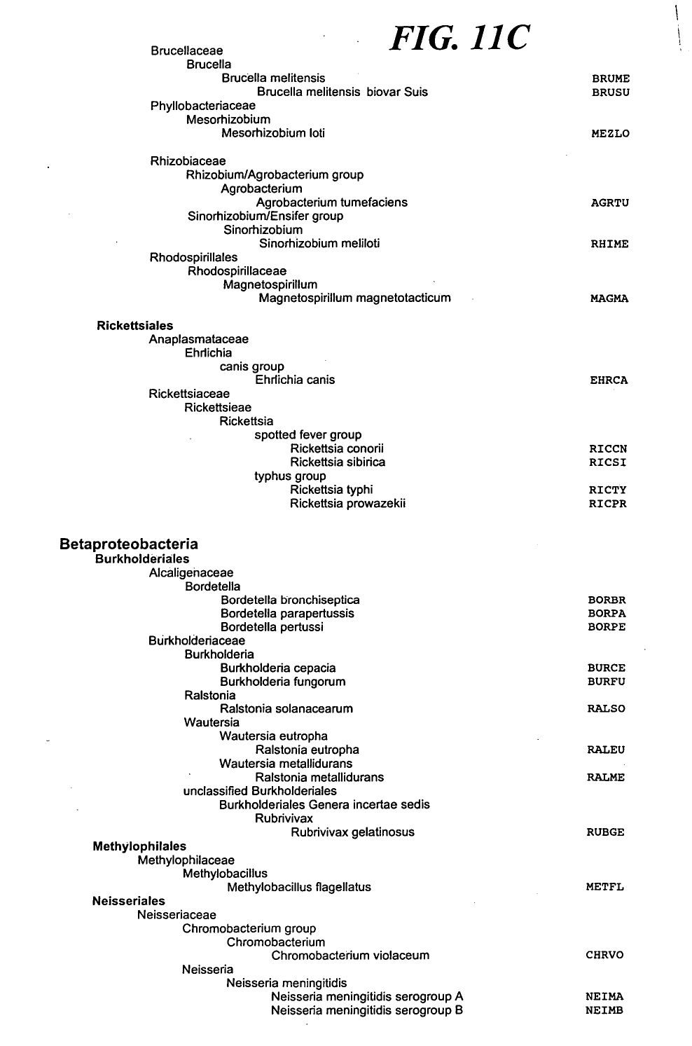

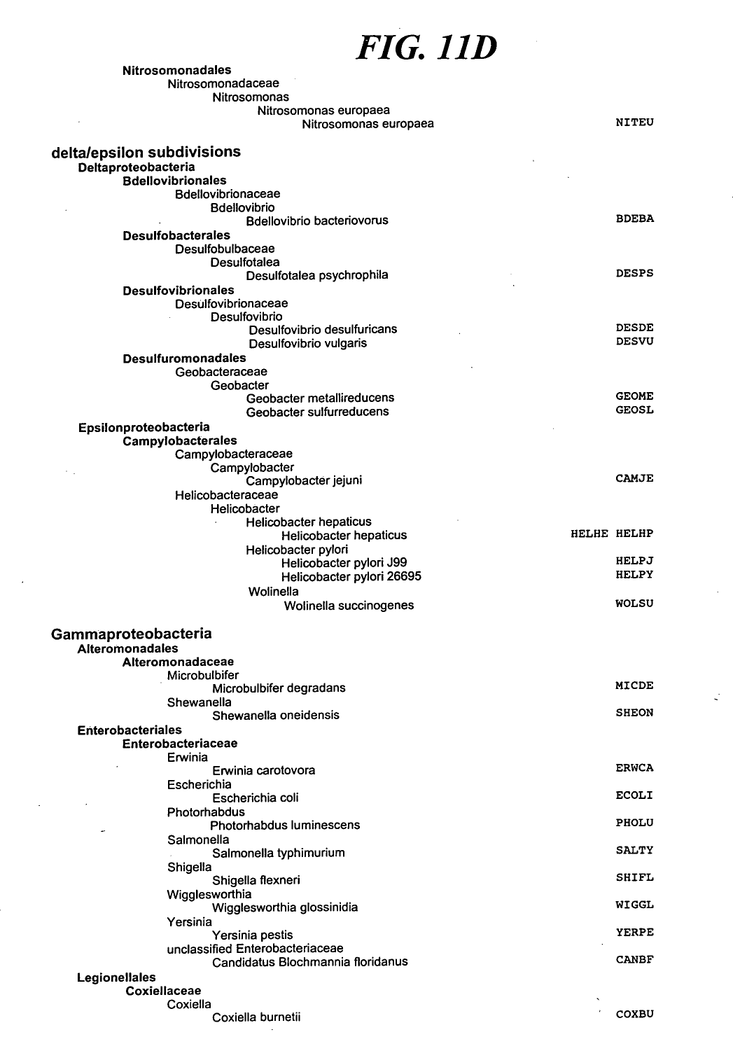

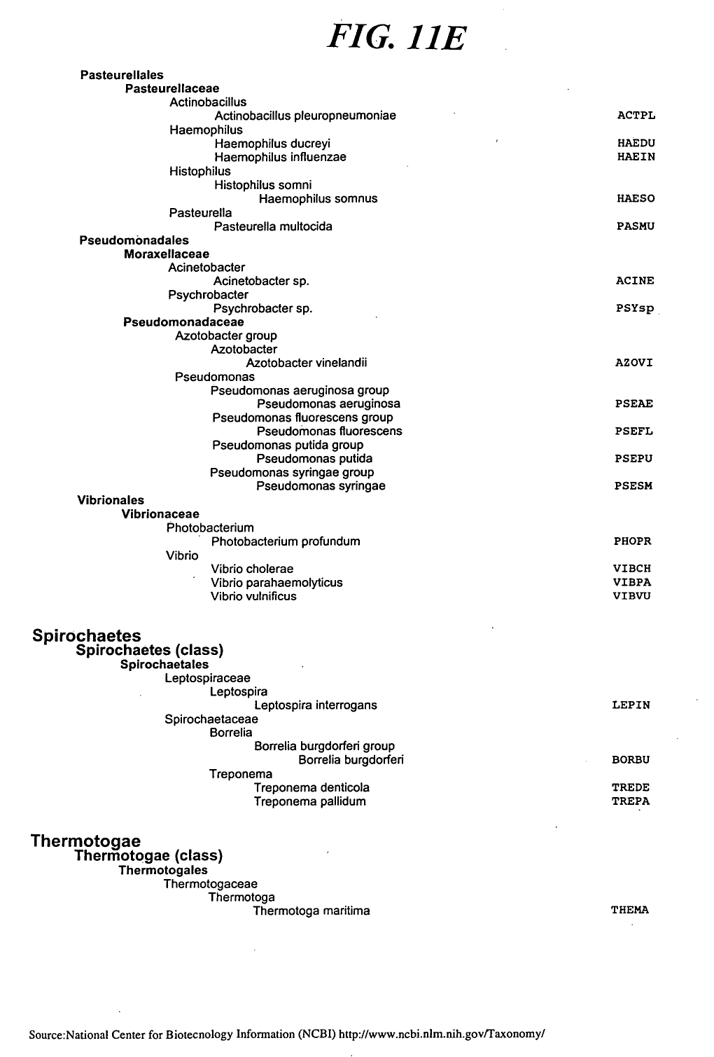

Structurally similar nucleic acid sequences encoding polypeptides having Lnt activity may be characterized by their ability to hybridize under stringent conditions to the native nucleic acid sequence, such as to SEQ ID NO: 1, described above or to polynucleotide sequences encoding Lnt proteins as described by FIGS. 9 and 10. Hybridization conditions may comprise hybridization in 5×SSC at a temperature of about 42 to 68° C. Washing may be performed using 2×SSC and optionally followed by washing using 0.5×SSC. For even higher stringency, the hybridization temperature may be raised to 68° C. or washing may be performed in a salt solution of 0.1×SSC. Other conventional hybridization procedures and conditions may also be used as described by Current Protocols in Molecular Biology, (1987-2004), see e.g. Chapter 2. Alternatively, variant nucleic acid sequences encoding polypeptides may be characterized by a particular degree of sequence similarity for instance, at least 60%, 70%, 80%, 90%, 95% or 99% similarity to the nucleic acid sequence of SEQ ID NO: 1.

Similarity may be determined by an algorithm, such as those described by Current Protocols in Molecular Biology, vol. 4, chapter 19 (1987-2004). Homology, sequence similarity or sequence identity of nucleotide or amino acid sequences may also be determined conventionally by using known software or computer programs such as the BestFit or Gap pairwise comparison programs (GCG Wisconsin Package, Genetics Computer Group, 575 Science Drive, Madison, Wis. 53711). BestFit uses the local homology algorithm of Smith and Waterman, Advances in Applied Mathematics 2: 482-489 (1981), to find the best segment of identity or similarity between two sequences. Gap performs global alignments: all of one sequence with all of another similar sequence using the method of Needleman and Wunsch, J. Mol. Biol. 48:443-453 (1970). When using a sequence alignment program such as BestFit, to determine the degree of sequence homology, similarity or identity, the default setting may be used, or an appropriate scoring matrix may be selected to optimize identity, similarity or homology scores. Similarly, when using a program such as BestFit to determine sequence identity, similarity or homology between two different amino acid sequences, the default settings may be used, or an appropriate scoring matrix, such as blosum45 or blosum80, may be selected to optimize identity, similarity or homology scores.

Protein binding assays: The interaction of a test compound with the Lnt protein may be determined by contacting polypeptides consisting of or comprising the periplasmic segments of the Lnt protein with the test compound. Compounds which bind to these portions of the Lnt protein are selected and may subsequently be tested for their ability to inhibit Lnt protein activity. For example, compounds which bind to the periplasmic segments between residues 28-33, 76-87 and 212-488 of Lnt may be contacted with bacteria expressing the Lnt protein, and Lnt protein activity may be determined. Methods of measuring Lnt protein activity are exemplified below, and include determining the accumulation of apolipoprotein, for example, in the cytoplasmic membrane, or determining the amount of lipoprotein appearing in the outer membrane or attached to the peptidoglycan and/or outer membrane.

Binding of test compounds to segments of the Lnt protein may be conducted using conventional binding assays, such as within microtiter plates which permit efficient screening of multiple compounds. Other analytic and Immunological methods suitable for such screening methods are described by Current Protocols in Molecular Biology (1987-2004), which is incorporated by reference, see e.g., Chapters 10 and 11.

Conditional mutants containing the lnt gene under the control of an inducible promoter may be used for determining the effect of a compound on Lnt activity. The ability to control the amount of Lnt produced by the cells used in the assay permits design of a more sensitive assay for Lnt activity, for example, by inducing only threshold amounts of Lnt to provide the highest sensitivity and discrimination between control and test sample. Moreover, these conditional mutants are useful for screening polypeptides expressed by other bacteria having N-acyltransferase activity by complementation methods.

Method for detecting Lnt enzyme activity. The effects of potential inhibitors the ability of Lnt to fatty acylate apolipoproteins are studied using apoLpp or other apolipoproteins purified from the Lnt-depleted E. coli mutant strain PAP8504. Enzyme activity is determined by the shift in migration from the apo to the mature lipoprotein form, as visualized by SDS-PAGE, by N-terminal protein sequencing or by mass spectrometry.

Methods for detecting and quantifying interactions between inhibitors and Lnt (or fragments thereof). Recombinant Lnt is purified as a complete protein (in detergent) or as the large periplasmic domain (in the absence of detergents) and enzyme activity is determined as above. The recombinant protein is then be immobilized on a mica chip and its interaction with test compounds analyzed by plasmon resonance using a Biacore or similar apparatus. Results are be validated or extended by flow equilibrium dialysis, gel filtration, affinity chromatography membrane filtration or ammonium sulfate precipitation, as appropriate.

Methods for screening for Lnt inhibitors in vivo. Mutant E. coli strain PAP8405 is used to define the effects of Lnt depletion (upon removal of arabinose from the culture) on cell physiology. The specific effect observed is the accumulation of apolipoproteins with a free N-terminal amine group. LipoCA-MalE(His)₆ (encoded by plasmid pCHAP1441) is purified from cells treated with (pools of) potential Lnt inhibitors and analyzed for the presence of the free amine group (by Adman degradation or other techniques). Less specific effects such as accumulation of lipoproteins in the inner membrane fraction or membrane fusion caused by defective transport of (apo)Lpp to the outer membrane are also used.

Lnt depletion (and, by inference, inhibition of Lnt by test compounds) causes an altered pattern of bacterial transcription as the bacteria (for example, E. coli) respond to the membrane stress caused by the accumulation of apolipoproteins and the consequent mislocalization or inactivation of specific membrane lipoproteins. A pattern of altered gene transcription specific to Lnt depletion (or inactivation) will be defined and selected genes will be fused to fluorescent reporter proteins. Test compounds will be incubated with bacteria expressing these gene fusions to identify those that induce the same pattern of altered transcription of the indicator genes as does Lnt depletion. The advantage of this system (called FLAIMES for Fluorescent Light As Indicator of Membrane and Envelope Stress) compared to other methods for detecting Lnt inhibition is that its potentially exquisite sensitivity allows even compounds that exert very weak effects (below the level that causes any gross phenotypic changes) to be identified.

### EXAMPLES

Materials and Methods

Strains, Plasmids and Construction of para-lnt Cassette

Strains of E. coli and S. enterica sv Typhimurium used in this study are listed in Table 3. Strain PAP8504 was constructed by homologous recombination according to the method developed by Wanner (30). The Kan-rpoCter-p_{araB} cassette was constructed by successive ligations between DNA fragments encoding the rpoCter gene, the kan gene or the araB promoter. Firstly, the terminator rpoCter was excised from pOM90 (31) using restriction enzymes EcoRI and KpnI and cloned in the plasmid pGP704 (32), at the same sites, giving pCHAP6560. Secondly, the gene encoding the kanamycin phosphotranferase (Kan) was PCR amplified using primers 5′-kan and kan-3′ (Table 5), from pBGS18 (33). The fragment was cloned into EcoRI and NotI sites upstream from the rpoCter gene in pCHAP6560 to create pCHAP6561. Finally, the araB promoter was PCR amplified using primers 5′-para and para-3′ (Table 5), from pBAD33 (34), and the fragment was cloned into XbaI and SalI sites downstream rpoCter gene in pCHAP6561 to create pCHAP6563. E. coli strain BW25113 carrying pKD46 was electroporated with 50 ng of the kan-rpoCter-p_{araB} fragment PCR amplified from pCHAP6563 using primers 5′-ybe-k and p-lnt-3′ (Table 5). These long primers include 45 nucleotides that hybridize with the ybeX or lnt genes and 19 or 25 priming nucleotides. PAP8504 was obtained after selection of transformants on agar containing kanamycin (25 μg/ml) and 0.2% arabinose, and was then incubated at 37° C. to eliminate the temperature-sensitive pKD46. The presence of the cassette in PAP8504 was verified by PCR amplification using primers 5′-ybe-k and p-lnt-3′ and by transduction into E. coli strain

PAP105 using phage P1 (35). Transductants were selected on agar containing kanamycin (25 μg/ml) and 0.2% arabinose.

Strain PAP8505 was obtained by transduction of lpp::Tn10 from an E. coli strain carrying this mutation (S. Gupta) using phage P1 and selection for resistance to tetracycline (16 μg/ml).

Plasmids used in this study are listed in Table 4. pCHAP6571, encoding LntEc, was constructed by ligating a DNA fragment, obtained by PCR amplification from DNA of strain MC4100 using primers 5′-cutE and cutE-3′ (Table 5), into EcoRI and BamHI sites in pUC18. The same procedure was employed to construct pCHAP6573 and pCHAP6574, encoding LntSe and LntSe(E435K) respectively, by using primers 5′-lnt and lnt-3′ (Table 5) and DNA from strains LT2 or SE5312. pCHAP6576 was obtained by site-directed mutagenesis of pCHAP6571 with the primer 5′-cutE435 (Table 5). Oligonucleotide-directed mutagenesis was performed using a Quickchange site-directed mutagenesis kit (Stratagene). A hexahistidine tag was added to the end of each of the six lipoMalE (14) constructs (Table 4) by replacing the 3′ BglII-HindIII fragment of the malE gene by the corresponding segment of the malE::His6 gene carried by pMalE-His (A. Davidson). Details of the construction of other plasmids listed in Table 4 are given below.

Growth Conditions

Liquid cultures were grown with aeration at 37° C. in Luria-Bertani (LB) medium (35), and cultures on plates were grown at 37° C. on LB agar, both supplemented with 0.2% arabinose, 0.4% glucose and/or 100 mM IPTG when necessary, and with appropriate antibiotics (100 μg/ml ampicillin, 25 μg/ml chloramphenicol, 50 μg/ml kanamycin).

For growth analysis and preparation of cell extracts, PAP8504 and PAP8505 were grown overnight in LB medium with 0.2% arabinose and washed in LB medium before being diluted 1:100 into LB medium with 0.2% arabinose or 0.4% glucose. Cells were grown at 37° C. with agitation to OD600 0.8 and then re-diluted 1:100 of fresh medium.

Immunoprecipitation of apoLpp

For radiolabeling, cells were grown in exponential growth in LB medium as above, washed and resuspended in minimal medium supplemented with 0.4% glucose or 0.5% glycerol (when cells were grown in medium containing arabinose) for 15 min at 30° C. Proteins from 1 ml of each culture were labeled with ³⁵S methionine for 5 min at 30° C., and then precipitated with 10% trichloroacetic acid. Proteins were immunoprecipitated with anti-Lpp (H. Tokuda) according to Kumamoto et al. (36), resuspended in 50 μl SDS sample buffer and analyzed by urea SDS-PAGE.

Construction and Analysis of lnt-lacZ and lnt-phoA Gene Fusions

The phoA gene encoding alkaline phosphatase PhoA, lacking the region coding for the signal peptide (from amino acid +13) was PCR amplified from plasmid pCHAP4020 (O. Francetic: unpublished) using primers 5′-phoA and phoA-3′ (Table 5) and cloned into PstI and HindIII sites in pBAD33 to obtain pCHAP6578. This plasmid was used to construct 9 plasmids, pCHAP6580 to pCHAP6588 (Table 4), encoding Lnt^{Ec}(30)-PhoA to Lnt^{Ec}(512)-PhoA (numbers in brackets correspond to the amino acid fusion site in Lnt^{Ec}). Each DNA fragment, obtained by PCR amplification with primers 5′-lnt^{Ec} and lnt^{Ec}30-3′ (or lnt^{Ec}53-3′, lnt^{Ec}80-3′, lnt^{Ec}117-3′, lnt^{Ec}154-3′, lnt^{Ec}190-3′, lnt^{Ec}218-3′, lnt^{Ec}476-3′, lnt^{Ec}513-3′ (Table 5)), was inserted into the XbaI and PstI sites in pCHAP6578. The same procedure was employed to construct the 9 plasmids, pCHAP6589 to pCHAP6597, encoding Lnt^{Ec}(30)-LacZ to Lnt^{Ec}(512)-LacZ. Firstly, the lacZ gene encoding beta-galactosidase LacZ (from amino acid +9) was PCR amplified from plasmid pRS552 (37) using primers 5′-lacZ and lacZ-3′ (Table 5) and cloned into the PstI and HindIII sites in pBAD33 to obtain pCHAP6577. An lnt^{Ec} DNA fragment obtained by PCR amplification with primers 5′-lnt^{Ec} and one of the nine 3′ primers lnt^{Ec}30-3′ to lnt^{Ec}512-3′ (Table 5), was inserted into the XbaI and PstI sites in pCHAP6577. Derivatives of strain KS272 producing the Lnt-PhoA and Lnt-LacZ chimeras or carrying pCHAP6577 or pCHAP6578 as controls were grown in LB medium at 30° C. supplemented with 25 μg/ml chloramphenicol and 0.2% arabinose. To measure alkaline phosphatase activity, cells were diluted in 1 ml of 50 mM Tris-HCl (pH 9.0) and then permeabilized by adding 50 μl of 10% octylpolyoxyethylene. After incubation at 37° C. for 5 min, the reaction was started by adding 100 μl of para-nitrophenylphosphate (10 mg/ml) and stopped with 500 μL of 1 M NaOH. To measure beta-galactosidase activity, the cells were diluted in Z-buffer (35) and permeabilized with octylpolyoxyethylene as above. The reaction was started by adding 200 μL of orthonitophenyl beta-D-galactopyranoside (4 mg/ml) and stopped by adding 500 μl of 1 M Na₂CO₃, Enzymes activities were calculated according to Miller (35) and are given in arbitrary units.

SDS-PAGE and Immunoblotting

Proteins solubilized in loading buffer were heated at 100° C. for 5 min and separated by SDS-PAGE in gels containing 9%, 10% or 12% acrylamide and, in some cases, 8 M urea to improve separation of apolipoproteins. Proteins were detected by staining with Coomassie blue or after transfer onto nitrocellulose membranes and incubation with primary polyclonal antisera to Lpp, SecG (W. Wickner), Pal (E. Bouveret), NlpD (S. Clarke), FhuA (M. Bonhivers), beta-galactosidase (Cappel), or alkaline phosphatase, and then by incubation with horseradish peroxidase (HRP)-conjugated secondary antiserum (Amersham Biosciences). His-tagged proteins were immunodetected using INDIA H is Probe-HRP (Pierce). Bound HRP-labeled antibodies were detected by enhanced chemiluminescence.

Separation of Plasma and Outer Membranes by Sucrose Flotation Gradient

One hundred ml of cultures at an OD 600 of 0.8-1.0 were collected by centrifugation and the pellet was resuspended in 10 ml of 25 mM HEPES (pH 7.4). Cells were disrupted by two passages through a French press (1,200 bar) and the lysate was supplemented with 10 μg/ml each of DNase I and pancreatic RNase A and then centrifuged for 10 min at 4,000 rpm to eliminate unbroken cells. Membranes were then collected by ultracentrifugation at 160,000×g for 1 hour at 4° C., resuspended and saturated at 60% (W/W) of sucrose in 200 μl of 25 mM HEPES (pH 7.4), and then placed at the bottom of a centrifuge tube. Steps (600 μl) were created using 56.2%, 53.2%, 50.2%, 47.1%, 44.2%, 41.2%, 38.1% and 35.9% sucrose solutions and the tubes were centrifuged in a swing-out rotor for 36 hours at 230,000×g at 10° C. Twenty fractions (250 μl) were collected from the top of the tubes and analyzed by SDS-PAGE and immunoblotting with appropriate antibodies. The concentration of sucrose in each fraction was determined from the refraction index.

Edman Degradation of Lipoproteins

To purify the histidine-tagged lipoMalE proteins for sequencing, bacteria from 100 ml of saturated LB broth culture containing 1 mM IPTG (to induce the expression of the malE gene, which is under p_{lacZ} control) were disrupted in a French Pressure Cell and the cell envelope was collected by centrifugation at 200,000×g for 60 min. Membrane proteins were dissolved in 2% 3-(N,N-dimethylmyristylammonio)propane sulfonate (Fluka) in 50 mM Tris-HCl (pH 8.0) and then adsorbed onto Talon cobalt affinity resin (Clontech) from which the lipoMalE^{His6} proteins were eluted with 200 mM imidazole. Purified proteins precipitated with 10% trichloroacetic acid and then separated by SDS-PAGE on 10% acrylamide gels and electroblotted onto Immobilon-PSQ PVDF membranes (Millipore) for automated sequencing.

Lnt Is Essential in E. coli

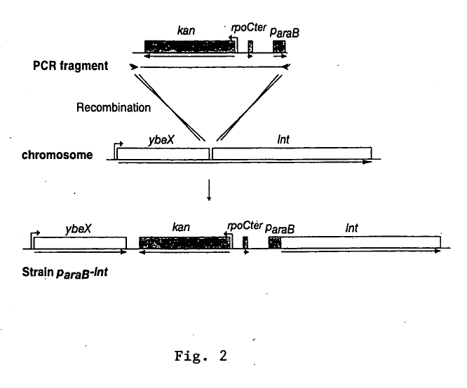

To study the consequences of Lnt depletion in E. coli, a conditional mutant was constructed in which the chromosomal lnt gene (originally called cutE (23), see Detailed Description) is expressed only from the arabinose-inducible AraC-dependent promoter p_{araB}. The lnt gene is located in an operon downstream from the ybeX gene, whose function is unknown. The 25 nucleotides between ybeX and lnt were replaced by a cassette containing a selectable kanamycin resistance gene (kan), the rpoC (rpoCter) transcription terminator (to prevent transcription read-though from pybeX) and p_{araB} (FIG. 2; see Materials and Methods), by homologous recombination. The resulting p_{araB}-lnt strain (PAP8504) ceased growth and began to lyse after 8 generations of growth in LB medium without arabinose and with glucose (to repress p_{araB} expression), and did not form colonies on minimal or LB agar (data not shown). The integration of the cassette between ybeX and lnt was first verified by PCR amplification using primers flanking the entire cassette and then by transduction into the E. coli strain PAP 105 using phage P1 with selection for the kanamycin-resistance cassette (see Materials and Methods). Donor and transductants exhibited the same arabinose-dependence and were complemented by a cloned copy of lnt expressed from placZ in a pUC18 derivative (pCHAP6571; see below), demonstrating that Lnt is essential for the viability of E. coli, as previously shown in S. enterica (22).

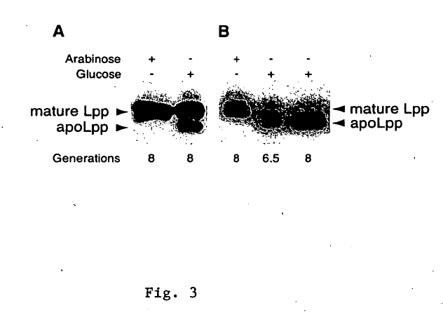

Immunoblotting with antibodies against the major outer membrane lipoprotein Lpp revealed the gradual appearance of the apolipoprotein form (apoLpp) when p_{araB}-lnt was repressed by glucose in LB cultures (FIG. 3A), confirming that the level of active Lnt declines under these conditions. ApoPal could not be detected by immunoblotting (data not shown; see Discussion). Immunoprecipitation of ³⁵S-methionine-labelled proteins with anti-Lpp revealed almost exclusive synthesis of apoLpp as the arabinose-deprived cultures approached the time at which they lyse (FIG. 3B). The analysis of lysozyme-treated peptidoglycan sacculi (38) revealed that both apo and mature forms of Lpp were peptidoglycan associated in Lnt depleted cells.

Lnt Depletion Induces Juxtapositioning of Plasma and Outer Membranes

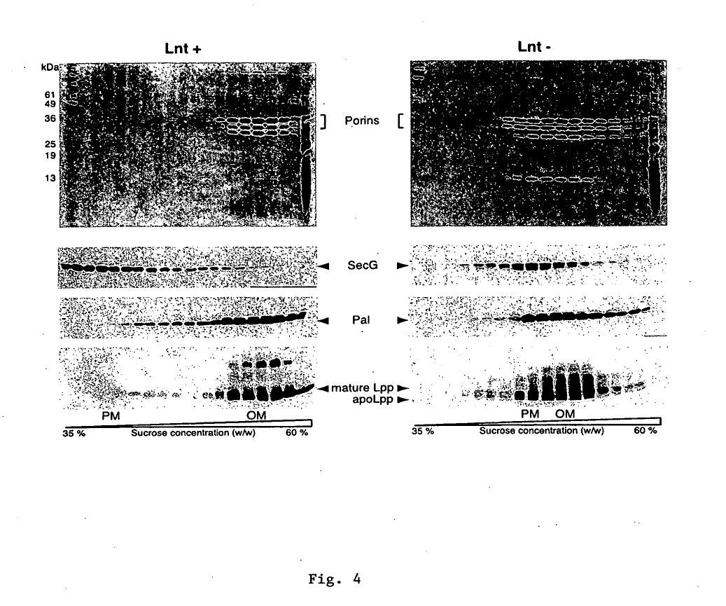

To analyze the localization of apolipoproteins, membranes from cells of strain PAP8504 grown in LB containing arabinose or glucose were separated by sucrose gradient centrifugation. Lpp and Pal in membranes from arabinose-grown cells were detected almost exclusively in dense fractions containing the outer membrane porins, well separated from less dense fractions containing SecG, an integral plasma membrane protein. In contrast, Lpp and apoLpp were both detected in the middle of the gradient, as were SecG, Pal and the porins, when membranes from Lnt-depleted cells (6.5 generations after repression of p_{araB}-lnt) were examined (FIG. 4). These data indicate that the architecture of the cell envelope is drastically altered by Lnt depletion, probably because the outer and plasma membranes become juxtaposed (see below) just before the onset of lysis.

Cell envelope instability was also observable by phase contrast microscopy of Lnt depleted E. coli, which were oval and swollen (data not shown). This phenotype was also reported for the S. enterica lnt^{ts} mutant SE5312 at 42° C. (22). Furthermore, sucrose flotation gradient analysis of membranes from this mutant grown at 42° C. revealed that plasma and outer membrane proteins were in the same fractions in the centre of the gradient, whereas these two classes of proteins were clearly separated in membranes from the wild-type strain (LT2) or from the mutant grown at 30° C. (data not shown). Thus, Lnt depletion in E. coli has the same effects on envelope architecture as Lnt inactivation in S. enterica. A similar association of plasma and outer membranes to that caused by Lnt depletion or inactivation was also shown to occur in E. coli after production of Lpp carrying the D+2 signal (29). It was proposed that this protein (LppDK) prevents the separation of the two membranes according to their density and causes lysis because it remains anchored in the plasma membrane and, nevertheless, is covalently linked to the peptidoglycan by its C-terminal lysine residue (29). Interestingly, introduction of an lpp::Tn10 mutation into strain SE5312 abolished temperature sensitivity caused by the lnt^{ts} mutation (22). Thus, a major factor contributing to the lethality caused by Lnt depletion could be the proposed retention of peptidoglycan cross-linked apoLpp in the plasma membrane (27).

To test this idea, the inventors analyzed twelve independent revertants of strain PAP8504 p_{araB}lnt) selected on LB agar without arabinose in a search for extragenic suppressor mutations in lpp. The presence of the kan-rpoCter-p_{araB} cassette in the revertants was verified by PCR using primers flanking the cassette, which was then transduced into strain BW25113 by P1 phage. Three of the mutants were resistant to P1 phage, probably due to changes in surface lipopolysaccharide composition. Six sets of transductants were arabinose-independent, suggesting that the donors had acquired a mutation in p_{araB} that rendered them AraC independent, while the remaining 3 sets of transductants were arabinose-dependent. Two of the last group of mutants were found to be devoid of Lpp when examined by SDS-PAGE and immunoblotting, whereas the third produced a form of Lpp that migrated aberrantly upon SDS-PAGE (slower migrating monomeric form and abundant dimeric form; not shown).

Sequence analysis revealed that the lpp gene in this mutant encodes a protein with glycine (G) to aspartate (D) substitution at position 14 in the signal peptide. Interestingly, the same mutation was previously identified in an E. coli K-12 mutant producing unmodified and unprocessed Lpp that was not cross-linked to the peptidoglycan and, therefore phenotypically Lpp− (39).

Finally, an lpp::Tn10 derivative of the p_{araB}-lnt strain PAP8504 was constructed by P1 transduction. This strain produced small colonies on LB agar (without arabinose), did not grow on LB agar containing glucose and, like the lpp+ parent strain, continued to grow for only 8 generations after replacement of arabinose by glucose in LB liquid cultures. When a wild-type allele of lpp was introduced on pJY111 (29), the transformants lost their ability to grow on agar in the absence of arabinose. In contrast, introduction of pJY151 (29), which carries an lpp allele encoding a variant of Lpp (LppSR) in which the C-terminal Lysine (K) that is normally cross-linked to the peptidoglycan, is replaced by arginine (R), still allowed growth on agar in the absence of arabinose (data not shown). These data confirm that the toxicity caused by failure to express p_{araB}-lnt can be partially relieved by preventing synthesis, export or cross-linking of Lpp to the peptidoglycan.

ApoLpp is Localized in the Plasma and Outer Membranes

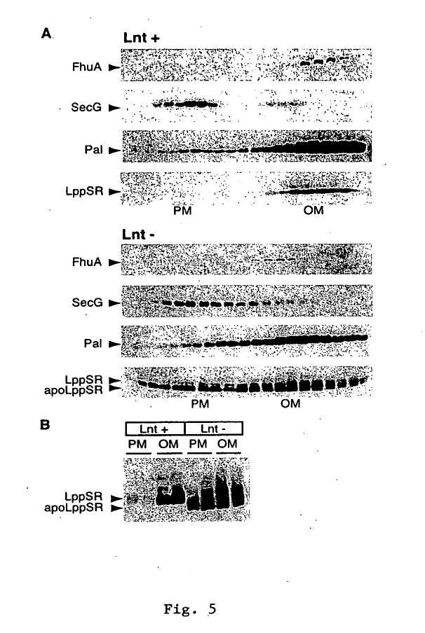

Although cells lacking Lpp are fragile and leak periplasmic proteins (5), their membranes can be separated by sucrose gradient centrifugation (not shown). Synthesis of LppSR did not affect this separation (FIG. 5A), allowing it to be used as a marker for localizing apolipoprotein after Lnt depletion. In this experiment, Lnt depletion by glucose repression of p_{araB}-lnt caused outer membrane proteins FhuA and Pal to appear in slightly less dense fractions than in membranes from arabinose-supplemented cultures, but they remained clearly distinct from the fractions containing the plasma membrane protein SecG. As already noted, apoPal could not be detected under these conditions (FIG. 5A). LppSR was detected in the outer membrane, while the apoLppSR was detected mainly in the plasma membrane fractions (FIG. 5A), though some apoLppSR also appeared in fractions enriched in outer membrane proteins (FIG. 5B).

Other Outer Membrane Lipoproteins also Accumulate in the Plasma Membrane when Lnt Levels are Depleted

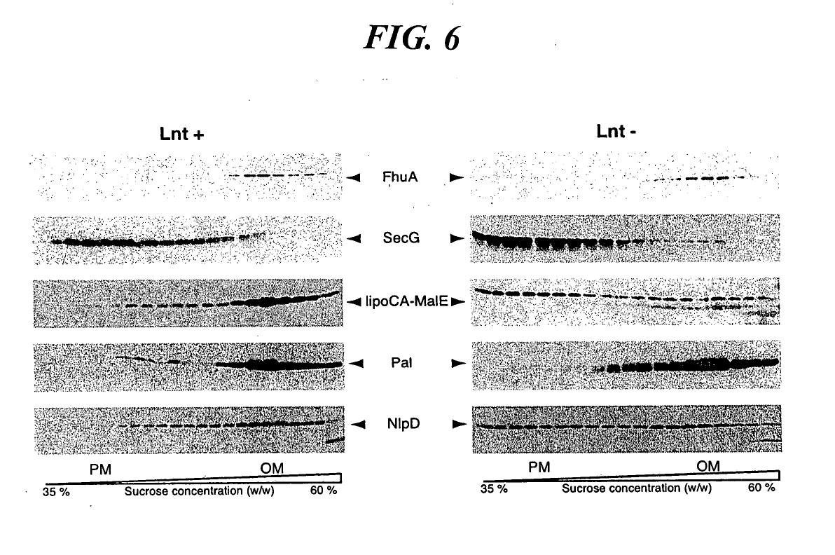

In the experiments described above, it was observed that plasma membrane accumulation of apoLpp but not of Pal upon Lnt depletion. To determine whether the localization of other outer membrane lipoproteins was affected by Lnt depletion, strain PAP8505 was transformed with pCHAP1447, encoding a fatty acylated variant of the E. coli periplasmic maltose binding protein MalE (14) with C-terminal hexahistidine extension. This protein (lipoCA-MalE) has a alanine residue at position +2 and is normally localized to the outer membrane (14). When strain PAP8505(pCHAP1447) was grown in glucose to deplete Lnt (Lnt− in FIG. 6), however, a substantial portion of lipoCA-MalE accumulated in the plasma membrane. Likewise, the endogenous outer membrane lipoprotein NlpD (40) accumulated in the plasma membrane under these conditions, whereas both proteins were located exclusively in the outer membrane in arabinose-grown, Lnt− replete (Lnt+ in FIG. 6) cells. As above, Pal was located exclusively in the outer membrane under both conditions. Pal from the outer membranes of Lnt+ and Lnt− cells could not be differentiated by SDS-PAGE under a variety of different conditions (not shown).

Alternative Plasma Membrane Retention Signals do not Operate by Impeding Lnt

Aromatic amino acids and proline at position +2 in the artificial lipoprotein, lipoMalE, cause its retention in the plasma membrane (14). To test whether these amino acids, unlike D+2 (27), function by impeding Lnt, the inventors determined whether the N-terminus of six different lipoMalE derivatives (with A+2, D+2, F+2 P+2, W+2 or Y+2) (14) was blocked to Edman degradation. The proteins were tagged with a C-terminal hexahistine that allowed them to be affinity purified from detergent solubilized envelope preparations (see Materials and Methods; MalE cannot be purified on amylose resin in the presence of detergents). All six proteins were found to have a blocked N-terminus, suggesting that none of the known plasma membrane retention signals operate by preventing N-acylation of the cysteine residue by Lnt, as already demonstrated for D+2 (27).

Characterization of the Mutation in lnt Gene of the S. enterica ts Mutant

Since the mutation in S enterica strain SE5312 that causes the temperature sensitive phenotype (22) was not characterized, the lnt gene from this mutant was PCR amplified and sequenced. A single mutation causing a glutamate (E) to lysine (K) substitution at position 435 was found in comparison with the lnt gene amplified from S. enterica strain LT2. Interestingly, E435 is located in a highly conserved region in the predicted hydrolase active site in Lnt^{Se}.

The same mutation was introduced in the E. coli K-12 lnt gene by directed mutagenesis, thereby producing the lnt(E435K) allele. Trans-complementation tests were then performed in S. enterica strain SE5312 and in E. coli PAP8504 with lnt or lnt(E435K) from E. coli or S. enterica cloned in high copy plasmids. Wild-type alleles of lnt^{Ec} (pCHAP6571) and lnt^{Se} (pCHAP6573) complemented both the lnt^{ts} mutation in strain SE5312 and the p_{araB}-lnt mutation in strain PAP8504, indicating that the Lnt enzymes from these two species are sufficiently similar (26 conservative substitutions and 28 non-conservative substitutions out of 512 residues) to be functionally interchangeable (Table 6).

The wild type strain LT2 did not grow on plates at 42° C. when Lnt^{Se}(E435K) (pCHAP6574) was expressed. Furthermore, the p_{araB}-lnt mutant PAP8504 failed to grow at either 37° C. or 42° C. when this protein was produced, confirming that the temperature sensitivity is caused by the E435K substitution in Lnt^{Se}. Interestingly Lnt^{Ec}(E435K) (pCHAP6576) also prevented growth of wild-type LT2 at 42° C., suggesting that overproduction of Lnt^{Ec}(E435K) or Lnt^{Se}(E435K) in LT2 is either toxic at 42° C. or is dominant negative over the activity of chromosome-encoded functional Lnt^{Se} at this temperature (Table 6). Lnt^{Ec}(E435K) and Lnt^{Se}(E435K) were not toxic at 42° C. in E. coli strain PAP8504 on LB agar containing arabinose, possibly because more Lnt is produced when lnt is expressed from p_{araB} in E. coli than when the lnt^{Se} is expressed from its own promoter in S. enterica. Moreover, in contrast to Lnt^{Se}(E435K), Lnt^{EC}(E435K) restored arabinose-independent growth of E. coli PAP8504 even at 42° C.

These data show that, even though Lnt^{Ec} and Lnt^{Se} proteins are functionally interchangeable, the E435K substitution resulted in a temperature-sensitive enzyme only in Lnt^{Se}, implying that other amino acid differences between these two proteins determine whether or not this substitution has an effect on enzyme activity or stability.

To distinguish between degradation and loss of activity of Lnt^{Se}(E435K) at 42° C., protein extracts of cells carrying pCHAP6574 were separated by SDS-PAGE and immunoblotted with monoclonal antibodies against the (His)6 tag at the C-terminus of the protein encoded by the cloned gene. The same amount of protein was detected in cells grown at 42° C. and 30° C. (data not shown), indicating that the E435K substitution specifically affects the activity of Lnt^{Se} at 42° C.

Plasma Membrane Topology of Lnt^{Ec}

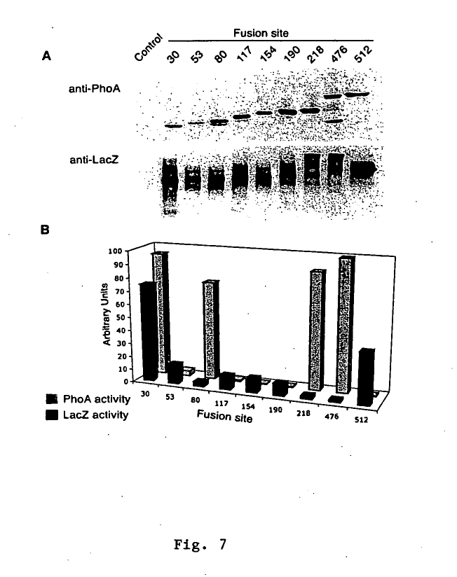

Bioinformatic analyses of lnt genes from Gram-negative bacteria predicted the presence of 8 segments of sufficient hydrophobicity to adopt a transmembrane topology ((Q10-A27)I, (W34-N50)II, (A57-V75)III, (P88-L112)IV, (W121-L138)V, (L163-L187)VI, (L195-1211)VII and (W489-L507)VIII; amino acid positions according to LntEc). This predicted topology of Lnt^{Ec} was tested by constructing a series of lnt-phoA and lnt-lacZ gene fusions. The lacZ and phoA genes (encoding beta-galactosidase LacZ and alkaline phosphatase PhoA, respectively) lacking their 5′ translation start signals (and, in the case of phoA, lacking a part of the region coding for the signal peptide) were PCR amplified and fused to 9 selected sites in regions of lnt^{Ec} encoding loops between the predicted transmembrane segments, in plasmids under the p_{araB} promoter. Each fusion was expressed in E. coli under arabinose induction and the activity of the reporter proteins was determined in permeabilized cells. Immunoblotting using anti-PhoA (alkaline phosphatase) and anti-LacZ (beta-galactosidase) antibodies revealed that the chimeras were stable and that their estimated sizes were as predicted from knowledge of the fusion site (FIG. 7A). Hybrid proteins with junction sites after Pro-53, Pro-117, Pro-154, Arg-190 and Lys-512 of Lnt^{Ec} showed high LacZ (>10 units) and low PhoA (<3 units) activities, indicating that these sites are exposed to the cytoplasm (PhoA is only active when exported to the periplasm, whereas LacZ is active only when retained in the cytoplasm; (41)). When the reporters were placed after Ala-80, Pro-218, and Pro-476, a reverse activity pattern (LacZ <3 units) and PhoA >75 units) was observed (FIG. 7B), indicating that these sites are exposed on the periplasmic side of the plasma membrane. However fusions constructed after Pro-30 of Lnt^{Ec} had high PhoA and LacZ activities (both >75 units). The N-terminal hydrophobic segment of Lnt presumably acts as a signal sequence to drive PhoA export, consistent with the presence of three arginine residues on its N-terminal (cytoplasmic) side (42), but cannot promote LacZ export, as reported previously for similar signal peptide-LacZ constructs (43). Similarly high enzyme activities were reported for other LacZ chimeras in which the fusion site was located after the first transmembrane segment of different E. coli polytopic plasma membrane proteins (44-46).

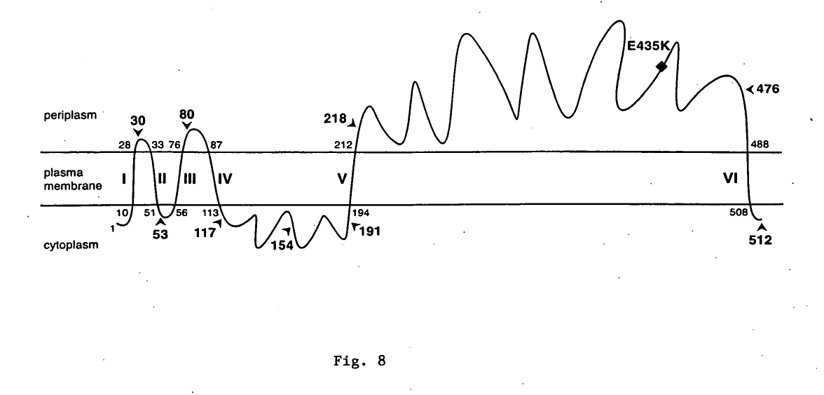

It is concluded that Lnt^{Ec} possesses 6 rather than the predicted 8 transmembrane segments interconnected by hydrophilic loops, with the N- and C-termini located in the cytoplasm (FIG. 8) and the predicted transmembrane segments between positions 112 and 195 being in the cytoplasm. This predicted topology is entirely consistent with von Heijne's positive-inside rule (42). According to this prediction, residue E435 is located in the large periplasmic loop between transmembrane segments 5 and 6.

Discussion

The data reported here provide in vivo evidence that Lnt-mediated N-acylation of major outer membrane lipoprotein Lpp and probably also of outer membrane lipoprotein NlpD and the artificial lipoprotein lipoCA-MalE is required for their efficient release from the plasma membrane. These data corroborate previous in vitro studies showing that LolA can not promote apoPal release from proteoliposomes by LolCDE (27). In view of the substantial structural differences between alternative lipoprotein plasma membrane retention signals (F+2, P+2, W+2 and Y+2) (14) and the canonical plasma membrane retention signal or Lol avoidance signal (D+2), it was speculated that one or all of them might operate by preventing N-acylation of normally outer membrane lipoproteins. The inventors demonstrated that this is not the case by showing the lipoMalE variants possessing these signals have blocked N-termini by Edman degradation, suggesting the presence of an N-acyl group. The way these amino acids prevent Lol-mediated lipoprotein transport to the outer membrane remains to be clarified. Even though apoLpp was clearly visualized after Lnt depletion, the apo form of another abundant outer membrane lipoprotein, Pal, could not be detected. The trivial explanation for this could be that the SDS-PAGE systems used are incapable of resolving apoPal and Pal, which differ by only 200 Da. It is also possible the apoPal has a higher affinity than apoLpp, apoNlpD and apolipoCA-MalE for Lnt and, therefore, that trace amounts of Lnt remaining after long periods of p_{araB}-lnt repression are sufficient for N-acylation of apoPal. This would also explain why Pal was only detected in outer membrane fractions after Lnt depletion, whereas apoLpp accumulated in the plasma membrane fractions (FIG. 5), and two other outer membrane lipoproteins, NlpD and lipoCA-MalE also accumulated in the plasma membrane when Lnt levels were depleted (FIG. 6). Alternatively, apoPal might be transported to the outer membrane by the Lol system, although this would be in contradiction to the observation that LolCDE releases Pal but not apoPal from proteoliposomes (27). ApoPal might also be unstable because it remains anchored in the plasma membrane or because its acylation is incomplete, although a plasma membrane anchored form of Pal with a D+2 is stable (data not shown). The plasma membrane apo-form of Lpp was found to be cross-linked to the peptidoglycan. This phenomenon explains the cofractionation of the plasma and outer membrane pools from envelopes of Lnt-depleted cells in sucrose gradients, since the peptidoglycan would be linked to both the outer membrane (via mature Lpp) and to the plasma membrane (via apoLpp). An identical phenomenon occurs when proLpp (uncleaved and unacylated) accumulates in the plasma membrane due to the absence of phosphatidyl glycerol caused by a mutation in the pgsA gene (47), or when Lpp (LppDK) is directed to the plasma membrane by a D+2 (29). In all cases, rupture of the bacteria in a French Press would result in the formation of outer and plasma membrane vesicles that are both linked to the peptidoglycan, causing them to float to an intermediate density in the sucrose gradients. The disorganization of the cell envelope that results from the mislocalization of peptidoglycan-bound Lpp (FIG. 4) is presumably a major but not the only cause of lysis that ensues Lnt depletion. Less dramatic changes in the density of the plasma and outer membrane fractions occur in Lnt-depleted cells in which Lpp is not linked to the peptidoglycan (LppSR; FIG. 5). These density shifts are attributed to the displacement of Lpp from the outer to inner membranes, which causes the density of the plasma membrane to rise and that of the outer membrane to decline. However, this scenario does not explain why apoLpp was also observed in fractions containing outer membrane proteins when Lnt was depleted (FIG. 5B). Bulk mixing of plasma and outer membrane can be ruled out because the integral plasma membrane protein SecG did not appear in the dense (outer membrane) membrane fractions (FIG. 5). Other methods of analysis must be sought to determine whether apoLpp retains limited ability to be transported to the outer membrane by the Lol system.

Elimination of Lpp allowed E. coli carrying the p_{araB}-lnt allele to grow on agar medium without arabinose but not in the presence of glucose. This observation suggests that other essential E. coli lipoproteins (LolB for example (48)) are inactivated when they are delocalized to the plasma membrane due to the absence of N-acylation (this study) or a functional Lol machinery (48,49). On the other hand, elimination of Lpp restored growth of the S. enterica lnt^{ts} mutant both on agar and in liquid culture at 42° C. (22), suggesting that S. enterica tolerates the combined removal of both Lnt and Lpp better than does E. coli and that the lnt^{ts} mutation does not completely inactivate Lnt^{Se} at 42°.

Mutations in lnt (cutE/actA) have been reported to confer copper sensitivity (23,24). This is an interesting observation, especially in view of the presence of a putative copper binding site, HFQMARM (amino acids 425-431 (23)), in the large periplasmic loop of Lnt between transmembrane segments 5 and 6. Only the first amino acids of this putative motif are highly conserved in Lnt from different bacterial species (as are the glutamine and arginine that precede and follow it, respectively), whereas the two methionines are often replaced by non-conservative amino acids. Moreover, the coding sequence of the mutant lnt (cutE) gene amplified from the copper-sensitive E. coli mutant reported by Rogers et al. (23) was found to be devoid of mutations (data not shown). This suggests that the mutation in this strain is not in lnt itself but might be in the upstream ybeX promoter. On the other hand, the transposon insertion in the R. meliloti lnt (actA) gene that causes copper and acid sensitivity would be expected to inactivate the gene, implying that Lnt function is not essential in Rhizobium. Interestingly, the mutation that confers the ts phenotype in S. enterica SE5312 (22) was found to cause a E435 to K substitution, 4 residues downstream from the putative copper binding motif in a large, highly-conserved periplasmic domain that might correspond to the active site. Despite all of these observations and other evidence linking lipoprotein biogenesis or the outer membrane lipoprotein NlpE to copper sensitivity (50,51), and the fact that some bacterial lipoproteins have copper binding sites (52,53), the role of Lnt in copper sensitivity remains unclear.

Escherichia coli Can Recover from Prolonged Lnt Depletion.

The previously-reported consequences of Lnt depletion, accumulation of apolipoproteins, membrane fusion and lysis (61, Robichon et al., 2005 and U.S. 60/623,241), were first detected 4, 6 and 8 generations approximately after removal of the arabinose induction of the para promoter that regulates lnt expression in the recombinant E. coli strains tested. To determine at which stage in this growth cycle viable cells could no longer be recovered when arabinose was reintroduced into the culture (to re-establish production of Lnt), arabinose-grown cultures in Luria-Bertani (LB) medium were washed to remove the arabinose, supplemented with glucose (to repress the para promoter) and inoculated at low cell densities (2-4×10⁷ bacteria/ml). Cultures were incubated with shaking at 37° C. and samples removed at time intervals to measure turbidity (as an indicator of growth) and colony formation on L agar containing glucose or arabinose. As expected, the cultures began to lyse (decline in turbidity) after 7-8 generations. Only low numbers of colonies were observed at all time points when the cells were plated on medium containing glucose. This was expected, since the repression of para-lnt expression would have been maintained irrespective of whether the cells were in liquid or on solid medium. The number of cells recovered on arabinose plates continued to increase exponentially until the culture started to lyse, at which point a progressive 4-log drop in recovery was recorded.

These data indicate that the progressive loss of Lnt causes structural rearrangements within the cell that are reversible as long as the cell remains viable. Presumably, the apolipoproteins that accumulate at later stages of Lnt depletion, before viability is lost, can be converted into mature lipoproteins and be correctly localized, once Lnt production is restored. Therefore, lysis is the cause of cell death that results from Lnt depletion. With respect to the use of Lnt as a potential drug target, these data indicate that the inhibitor must be present in sufficient quantities to saturate and inactivate all copies of Lnt in the cell and all copies made thereafter. It should be stressed however, that the effects of an inhibitor of Lnt action are likely to be much more rapid than the inhibition of Lnt production (in which the enzyme must be diluted to below a crucial level before effects are observed).

Shock Responses Induced Upon Lnt Depletion.

E. coli mounts a number of well-characterized stress reactions in response to changes in its envelope structure, such as accumulation of misfolded proteins or defects in protein localization. Two of the main stress responses involve a specific sigma factor, sigma E or a two-component regulatory system called Cpx (60, Raivio, 2005). To determine whether these stress responses are induced by Lnt depletion, and if so, at what stage in the growth cycle, the para-lnt construct was transduced by P1 phage transduction into strains carrying degP-lacZ, dsbA-lacZ and fpkA-lacZ fusions (controlled, respectively, by Cpx, sigma E and both; strains supplied by Prof T. Silhavy, Princeton University, USA), in which lacZ expression (measured as beta-galactosidase activity) indicates the level of induction of the corresponding stress gene. Cells were first grown in LB medium with arabinose, then washed and resuspended in fresh LB medium containing arabinose or glucose (induction or repression of para-lnt, respectively) and incubated until lysis occurred (in the glucose-supplemented culture; i.e., 6.5-8 later). A typical result from these experiments is shown in Table 1.

TABLE 1
Expression of Cpx-controlled degP-lacZ following depletion
of Lnt levels by switching from arabinose induction to
glucose repression in E. coli carrying para-lnt.

| Time | | beta- | | beta- |
|---|---|---|---|---|
| (min- | ARABINOSE | galactosidase | GLUCOSE | galactosidase |
| utes) | OD600 nm | (units/ml) | OD600 nm | (units/ml) |
| 0 | 0.06 | | 0.06 | |
| 45 | 0.08 | 17 | 0.08 | 12 |
| 80 | 0.17 | 17 | 0.18 | 15 |
| 125 | 0.46 | 39 | 0.56 | 49 |
| 145 | 0.96 | 100 | 1.20 | 299 |
| 175 | 1.48 | 167 | 1.72 | 999 |
| 195 | 1.88 | 231 | 2.08 | 2170 |
| 220 | 2.32 | 356 | 2.28 | 3326 |
| 245 | 2.88 | 553 | 1.52 | 5600 |
| 285 | 3.32 | 430 | 1.16 | 7833 |

In this experiment, increased Cpx stress response was recorded in the glucose-grown culture 145 minutes (4 generations) after removal of arabinose, indicating that disorganization of envelope structure occurred at this early time point. Massive induction of the Cpx response occurred as the cells began to grow more slowly (175-195 minutes) and to lyse (after 220 minutes). Identical response curves were obtained for the dsbA-lacZ and fpkA-lacZ fusions, indicating that both Cpx and sigma E stress responses are induced upon Lnt depletion. The so-called phage shock response, which also responds to envelope defects (54, Brissette et al., 1990) and was monitored here using a pspA-lacZ fusion or by immunoblotting to measure the level of PspA protein in the cells, was not induced after arabinose removal (data not shown), indicating that Lnt depletion induces a specific spectrum of shock responses. Monitoring of the Cpx and sigma E shock responses could be included in screens for inhibitors of Lnt.

Search for Suppressors of Lnt Depletion.

In previous studies, it was (55, Gupta et al., 1993) noted that inactivation of the lpp gene encoding the major outer membrane lipoprotein allowed E. coli and S. enterica to survive the effects of Lnt depletion or a ts mutation in the lnt gene, respectively, better than when the lpp gene was intact. However, it was also noted that Lnt depletion was still lethal and caused lysis even of strains devoid of Lpp under severe repression conditions (growth in glucose). The inventors also noted that survivors of the double mutant (para-lnt, lpp::Tn10) were obtained on LB plates containing glucose (severe repression conditions). These colonies proved to be of two types: either they did not grow when restreaked on glucose-containing medium and lysed when grown in liquid medium containing glucose, indicating that they do not carry a true suppressor mutation, or they could not be reisolated as single colonies (due to slow growth and the presence of extensive capsular slime). It was concluded that no single or pair of mutations is likely to overcome the effects of an inhibitor of Lnt action.

Lnt from Other Bacteria can Replace Lnt in E. Coli.

As a first step towards characterizing Lnt in bacteria other than E. coli and S. enterica, the inventors selected strains from a list of bacteria with putative Lnt homologues (see FIGS. 9 and 10), designed specific oligonucleotides and cloned the corresponding gene into an expression vector (pUC) under placZ control that was then transformed into E. coli carrying the para-lnt allele. Cells were grown on plates containing glucose to determine whether expression of the foreign lnt gene could overcome the effects of repression of the endogenous lnt gene. Wherever possible, a hexahistidine tag was added to the C-terminus of the recombinant protein (by incorporation of 6 histidine codons into the oligonucleotide used to amplify the gene) to permit verification of expression of the heterologous gene in E. coli. The strains used as sources of DNA for these experiments fell into two groups: Gram-negative bacteria and high G+C Gram-positive bacteria that, unlike the low G+C Gram-positive bacteria, appear to have an lnt homologue. Cloned genes were verified by sequencing and differences in the sequence of the predicted protein compared to that of the strain in which the gene was initially sequenced were noted. The data obtained in these experiments are summarized in Table 2.

TABLE 2

| | | | Extra | | | | Growth | Growth | Growth | Growth |
|---|---|---|---|---|---|---|---|---|---|---|
| | | 6× | N-term | DNA | Mutation | His Tag | 30° C. | 30° C. | 37° C. | 37° C. |
| Plasmid | lnt^{a} | His | AA^{d} | Sequence | Site | Detected | Ara | Glu | Ara | Glu |
| pCHAP6571 | Ec | Yes | | wt | | Yes | + | ++ | ++ | ++ |
| pUC18 | — | | | — | | No | + | − | ++ | − |
| pCHAP6601 | Yp | No | 8 | wt | | absent | + | + | ++ | ++ |
| pCHAP6603 | Pa | Yes | 6 | wt | | Yes | + | + | −^{b} | ++ |
| pCHAP6605 | Cg | Yes | 8 | wt | | Yes | + | − | +^{c} | − |
| pCHAP6607 | Nm | Yes | 6 | Q72→R | TM₃^{a} | Yes | + | + | ++ | ++ |
| pCHAP6609 | Sl | Yes | 6 | No genome | | Yes | + | − | ++ | − |
| | | | | sequence | | | | | | |
| pCHAP6611 | Sc | Yes | 6 | A233→V | PP | Yes | + | − | ++ | − |
| | | | | T401→P | domain | | | | | |
| pCHAP6613 | Vc | Yes | 11 | wt | | nd | + | + | ++ | ++ |
| pCHAP6628 | Bp | No | 6 | Not sequenced | | Absent | + | + | nd | nd |

^{a}EC, E. coli K-12; Yp, Yersinia pestis; Pa, Pseudomonas aeruginosa; Cg, Corynebacterium glutamicum; Nm, Neisseria meningitidis; Sl, Streptomyces lividans; Sc, S. coelicolor; Vc, Vibrio cholerae; Bp, Bordetella pertussis
^{b}Pa lnt causes inhibition of growth at 37° C. but not at 30° C. in media containing arabinose
^{c}Cg lnt causes small colony growth at 37°C. but not at 30° C. in media containing arabinose.
^{d}Additional amino acids come from the cloning site used to fuse the gene directly in-frame with the lacZ translation start site in the pUC vector.
nd, not determined

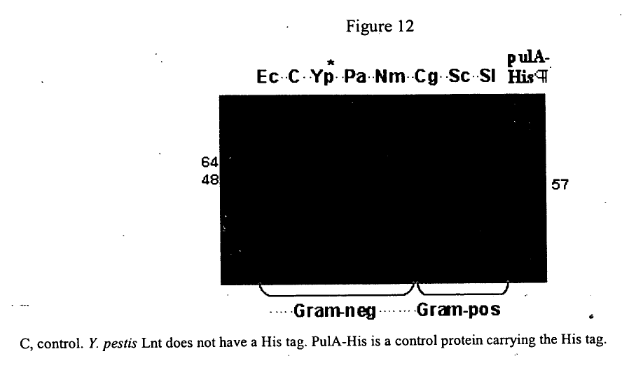

In general, the lnt genes cloned from the Gram-negative bacteria (Yersinia, Vibrio, Neisseria, Bordetella) were able to replace the E. coli lnt gene (grown in medium containing glucose in Table 2), although expression of the P. aeruginosa lnt gene was apparently toxic at 37° C. In contrast, none of the three lnt genes cloned from the Gram-positive bacteria (Streptomyces and Corynebacterium) were able to do so, despite the fact that the products of the genes could be detected by immunoblotting with antibodies against the histidine tag (FIG. 12). Intriguingly, these proteins all migrated more slowly than their counterparts from Gram-negative bacteria, despite the fact that sequence predictions indicate that they have a similar molecular mass.

The data shown above and in FIG. 12 show that Lnt proteins in several Gram-negative bacteria are functionally interchangeable. The reason for the failure of the Lnt proteins from Gram-positive bacteria to function in this assay remains to be explored.

lnt Gene is Apparently Essential in Other Gram-Negative Bacteria

Several laboratories are currently engaged in systematic analysis of gene requirements in Gram-negative bacteria, either by random transposon mutagenesis or directed gene inactivation. In all cases where the inventors have access to the data, these studies failed to inactivate the lnt gene, indicating that it is probably essential, as expected from the known properties of lnt in E. coli and S. enterica.

Two Sets of Lnt Homologues

Continued bioinformatic analysis of Lnt homologues has resulted in the division of this group of proteins into two sets. The first comprises true Lnt homologues. They are all found in exclusively Gram-negative bacteria and high G+C Gram-positive bacteria (Corynebacteria, Mycobacteria, Streptomyces, etc). All of these proteins are predicted to be associated with the plasma membrane with a substantial segment facing the outside (Gram-positive bacteria) or periplasm (Gram-negative bacteria). The predicted topologies of all of these proteins are similar to that determined experimentally for the E. coli Lnt (61, Robichon et al., 2005), i.e., 6 transmembrane segments with the N- and C-termini in the cytoplasm. Residues both within the large periplasmic loop and elsewhere within the protein are conserved (in future studies, these residues will be targeted by site-directed mutagenesis of the E. coli protein to establish their role in catalysis; see below).

The second subclass of proteins is homologous only to the large periplasmic loop of E. coli Lnt. These proteins do not have any segment of high hydrophobicity that could target them to the membrane (as a signal sequence) or anchor them in the membrane. They are found in a wide range of organisms ranging from bacteria through fungi and plants to man. Several of these proteins have been characterized at the molecular level. Generally, they are all described as hydrolases, and their activities include nitrilases, aliphatic amidases, cyanide hydrolases and beta-alanine synthases. Characteristically, they all have an active site cysteine residue that is conserved in Lnt in the conserved motif GXXI/VCVE/D. Structures of several of these proteins have been determined at high resolution (57, Kumaran et al., 2003; 58, Nakai et al., 2000; 59, Pace et al., 2000; 62, Wang et al., 2001). They all reveal similar folds and positioning of conserved residues that are also found in Lnt.

These data suggest that Lnt operates in a similar fashion to these hydrolases. The catalysis of fatty acid transfer to lipoproteins presumably occurs in two steps: hydrolysis of phospholipids to release the mainly palmitoyl fatty acid (hydrolase step), followed by transfer and ligation to the exposed amine group on the apolipoprotein (56, Jackowski and Rock, 1986). The large periplasmic loop of Lnt is conserved in the hydrolases, indicating that it probably performs the first step in this reaction, i.e., cleavage of the N1-linked fatty acid on phosphatidyl ethanolamine. If this is the case, then the second step must be performed by a second catalytic domain (or, potentially, by another, hitherto unidentified enzyme). Site directed mutagenesis of residues important for this activity in Lnt (or inactivation of the gene coding for this enzyme) would result in uncontrolled hydrolysis of PE and accumulation of free palmitate in the membrane. Residue E435 known to be essential for full activity of S. enterica Lnt (61, Robichon et al., 2005) is located in the presumed hydrolase domain of Lnt.

Cell Lines Deposited Under the Terms of the Budapest Treaty

The following cell lines were deposited at the C.N.C.M. (Collection Nationale de Cultures de Microorganismes) on Oct. 22, 2004 under the terms of the Budapest Treaty:
PAP8504 (CNCM I-3310), Escherichia coli strain K-12 with a chromosomal copy of the lnt gene under exclusive control of the arabinose-AraC promoter.
PAP8505 (CNCM 1-3311), Escherichia coli strain derived from PAP8504 strain carrying an insertion of Tnt10 transposon in the chromosomal lpp gene.
PAP105 (pCHAP1441)(CNCM I-3312), Escherichia coli strain K-12 PAP105 carrying vector PCHAP1441. The vector pCHAP1441 is a plasmid BGS18+ carrying an artificial gene plac-pulA-CAmalE. This artificial gene is constituted by the lac promoter and the coding sequence of malE gene modified so as:

the native signal peptide of malE is replaced by the signal peptide and the first four amino acids (CDNS) of the PulA protein;

the native amino acid (aspartic acid or D) in position +2 of the PulA protein is replaced by an alanine (A); and

six histidines are added at the C-terminal extremity of the protein.

Specific embodiments of the invention are described below. An isolated or purified polypeptide comprising one Lnt periplasmic segment selected from the group consisting of sequences SEQ ID NO: 3, 4 and 5, sequences which consist essentially of amino acids residues 28-33, 76-87 or 212-488 of SEQ ID NO: 2, or a fragment thereof. Such a polypeptide may consist of a fragment from 5 up to 250 amino acids residues or any intermediate size within this range, such as at least 10, 20, 25, 50, 100, 125, 150, 200 or 225 residues. It may specifically comprise the Lnt periplasmic segments described by SEQ ID NO: 3, SEQ ID NO: 4 or SEQ ID NO: 5, or may consist essentially of residues 28-33, 76-87 or 212-488 of SEQ ID NO: 2.

The polypeptides described above may be produced or expressed by a recombinant cell comprising a polynucleotide encoding at least one periplasmic segment of the Lnt protein or a lnt gene, especially when the polynucleotide encoding the periplasmic segment is under the control of an inducible promoter. For example, a cell may be transformed with the polynucleotide of SEQ ID NO: 1 or a polynucleotide encoding residues 28-33, 76-87 or 212-488 of SEQ ID NO: 2 or a fragment thereof, such as a fragment having a lnt or Lnt activity. Such cells may be eukaryotic cells or prokaryotic cells, such as gram-negative bacteria, and more specifically Escherichia coli.

A suitable inducible promoter may be selected from those known in the art. An arabinose-AraC-dependent promoter may be used. One example of such a cell is E. coli strain PAP8504 deposited under accession number I-3310 at CNCM or E. coli strain PAP8505 deposited under accession number I-3311 at the CNCM.

Isolated or purified polynucleotides encoding one or more Lnt protein domains are also disclosed in FIGS. 9 and 10. The complements of such polynucleotides are also specifically described based on the known complementary of nucleotides and functional fragments of such polynucleotides or their complements are encompassed by the present invention. Such polynucleotides may range from 15 up to 750 nucleotides in length or any intermediate range or value within this range, such as 25, 50, 100, 200, 250, 400, 500, 600 or 700 nucleotides. Such polynucleotides may encode the lnt periplasmic domains such as those described by SEQ ID NO: 3, SEQ ID NO: 4 or SEQ ID NO: 5, or may consist essentially of residues 28-33, 76-87 or 212-488 of SEQ ID NO: 2. They may also encode sequences encoded by SEQ ID NO: 1 or a sequence which is able to hybridize under stringent conditions to SEQ ID NO: 1 or its complement and which encodes a polypeptide having N-acyltransferase activity.

Such polynucleotides or their complements or functional fragments may be inserted into a suitable vector or host cell by methods known in the art.

Compounds which bind to Lnt protein may be screened or identified by contacting comprising contacting a test compound with an isolated or purified polypeptide comprising one Lnt periplasmic segment, such as Lnt periplasmic segments selected from the group consisting of sequences SEQ ID NO: 3, 4 and 5, sequences which consist essentially of amino acids residues 28-33, 76-87 or 212-488 of SEQ ID NO: 2, or a fragment thereof Such a method may involve determining the amount of binding of said compound to said polypeptide.

Compounds which modulate the activity of Lnt protein, for example, increase or decrease its activity, may also be identified or screened by contacting a test compound with a cell expressing a polypeptide comprising at least one periplasmic segment of the Lnt protein, and determining the amount of apolipoprotein which accumulates in the inner membrane and/or the amount of lipoprotein in the outer membrane and/or associated with the peptidoglycan. Cpx and sigma E shock responses may be used as cell viability determinants to identify a compound which inhibits the activity of Lnt protein.

Compounds which modulate the activity of Lnt protein may also be characterized or identified by contacting a cell expressing a polypeptide comprising one periplasmic segment of the Lnt protein with a test compound and determining membrane integrity or cell viability compared to a cell not contacted with said test compound.

A bacterial gene coding for a protein functionally equivalent to Lnt, may be identified or characterized by introducing the bacterial gene into a cell expressing a lnt periplasmic domain under the control of an inducible promoter in culture conditions where the inducible promoter is off, scoring the colonies and concluding that the bacterial gene encodes a protein functionally equivalent to Lnt if there are more colonies with the introduced bacterial gene than without.

The invention also covers the particular cell lines PAP8504 (CNCM I-3310), PAP8505 (CNCM I-3311) and PAP105 (pCHAP1441)(CNCM I-3312).

A technical platform comprising a least standard quantities of an isolated or purified polypeptide comprising one Lnt periplasmic segment, such as those selected from the group consisting of sequences SEQ ID NO: 3, 4 and 5, or sequences which consist essentially of amino acids residues 28-33, 76-87 or 212-488 of SEQ ID NO: 2, or a fragment thereof, reagents for testing the N-acyltransferase activity, and reagents for testing the amount of apolipoprotein which accumulates in the inner membrane and/or the amount of lipoprotein in the outer membrane and/or associated with the peptidoglycan.

A technical platform comprising at least a recombinant cell expressing a polypeptide comprising one Lnt periplasmic segment, such as those selected from the group consisting of sequences SEQ ID NO: 3, 4 and 5, or sequences which consist essentially of amino acids residues 28-33, 76-87 or 212-488 of SEQ ID NO: 2, or a fragment thereof, reagents for testing the N-acyltransferase activity, and reagents for testing the amount of apolipoprotein which accumulates in the inner membrane and/or the amount of lipoprotein in the outer membrane and/or associated with the peptidoglycan.

### MODIFICATIONS AND OTHER EMBODIMENTS

Various modifications and variations of the described compositions and their methods of use as well as the concept of the invention will be apparent to those skilled in the art without departing from the scope and spirit of the invention. Although the invention has been described in connection with specific preferred embodiments, it should be understood that the invention as claimed is not intended to be limited to such specific embodiments. Various modifications of the described modes for carrying out the invention which are obvious to those skilled in the biological, molecular biological, biochemical, chemical, medical, pharmacological, pharmaceutical, or related fields are intended to be within the scope of the following claims.

### INCORPORATION BY REFERENCE

Each document, publication, patent application, patent publication or patent cited by or referred to in this disclosure is incorporated by reference in its entirety. However, no admission is made that any such reference constitutes prior art and the right to challenge the accuracy and pertinency of the cited documents is reserved. This application claims priority under 35 U.S.C. 119(e) to U.S. Provisional Application 60/609,241, filed Sep. 14, 2004 and 60/623,860, filed Nov. 2, 2004, both of which are hereby incorporated by reference.

TABLE 3
Bacterial strains.

| | | Source/ |
|---|---|---|
| Strain | Characteristics | reference |
| BW25113 | E. coli K-12 lacIq rrnBT14 | (30) |
| | γlacZ_{WJ16} hsdR514 | |
| | γaraBAD_{AH33} γrhaBAD_{LD78} | |
| PAP8504 | BW25113 ybeX-(kan-rpoCter-p_{araB})-lnt | This study |
| | This study | |
| PAP8505 | PAP8504 lpp::Tn10 | This study |
| PAP105 | E. coli K-12 γlac-pro) F′ | Lab collection |
| | (lacl^{q} γlacZM15 pro⁺ Tn10) | |
| KS272 | E. coli K-12 βlacZX74 galE | K. Strauch via |
| | galK thi rpsL γphoA | G. Georgiou |
| LT2 | S. enterica sv Typhimurium | Lab collection |
| SE5312 | S. enterica sv Typhimurium | (22) |
| | lnt^{ts} leu485 | |

TABLE 4
Plasmids.

| Plasmid | | Resis- | Source/ |
|---|---|---|---|
| number | Characteristics | tance | reference |
| pKD46 | araC repA101ts p_{araB}-γ-γ-exo | Ap | (30) |
| pJY111 | ptac-lpp | Ap | (29) |
| pJY151 | ptac-lppSR | Ap | (29) |
| pCHAP1447 | pSU18+ p_{lac}-pulA-CAmalE | Cm | pCHAP4585(14) |
| pCHAP1441 | pBGS18+ p_{lac}-pulA-CAmalE | Km | pCHAP4585(14) |
| pCHAP1442 | pBGS18+ p_{lac}-pulA-CDmalE | Km | pCHAP4587(14) |
| pCHAP1443 | pBGS18+ p_{lac}-pulA-CFmalE | Km | pCHAP4589(14) |
| pCHAP1444 | pBGS18+ p_{lac}-pulA-CPmalE | Km | pCHAP4597(14) |
| pCHAP1445 | pBGS18+ p_{lac}-pulA-CWmalE | Km | pCHAP4603(38) |
| pCHAP1446 | pBGS18+ p_{lac}-pulA-CYmalE | Km | pCHAP4604(14) |
| pCHAP6560 | pGP704-rpoCter | Ap | This study |
| pCHAP6561 | pCHAP6560-kan | Ap | This study |
| pCHAP6563 | pCHAP6561-p_{araB} | Ap | This study |
| pCHAP6571 | pUC18− p_{lac}-lnt^{Ec} | Ap | This study |
| pCHAP6573 | pUC18− p_{lac}-lnt^{Se} | Ap | This study |
| pCHAP6574 | pUC18− p_{lac}-lnt^{Se}(E435K) | Ap | This study |
| pCHAP6576 | pUC18− p_{lac}-lnt^{Ec}(E435K) | Ap | This study |
| pCHAP6577 | pBAD33-p_{araB}-lacZ | Cm | This study |
| pCHAP6578 | pBAD33-p_{araB}-phoA | Cm | This study |
| pCHAP6580 | pBAD33-p_{araB}-lnt^{Ec}30-phoA | Cm | This study |
| pCHAP6581 | pBAD33-p_{araB}-lnt^{Ec}53-phoA | Cm | This study |
| pCHAP6582 | pBAD33-p_{araB}-lnt^{Ec}80-phoA | Cm | This study |
| pCHAP6583 | pBAD33-p_{araB}-lnt^{Ec}117-phoA | Cm | This study |
| pCHAP6584 | pBAD33-p_{araB}-lnt^{Ec}154-phoA | Cm | This study |
| pCHAP6585 | pBAD33-p_{araB}-lnt^{Ec}190-phoA | Cm | This study |
| pCHAP6586 | pBAD33-p_{araB}-lnt^{Ec}218-phoA | Cm | This study |
| pCHAP6587 | pBAD33-p_{araB}-lnt^{Ec}476-phoA | Cm | This study |
| pCHAP6588 | pBAD33-p_{araB}-lnt^{Ec}512-phoA | Cm | This study |
| pCHAP6589 | pBAD33-p_{araB}-lnt^{Ec}30-lacZ | Cm | This study |
| pCHAP6590 | pBAD33-p_{araB}-lnt^{Ec}53-lacZ | Cm | This study |
| pCHAP6591 | pBAD33-p_{araB}-lnt^{Ec}80-lacZ | Cm | This study |
| pCHAP6592 | pBAD33-p_{araB}-lnt^{Ec}117-lacZ | Cm | This study |
| pCHAP6593 | pBAD33-p_{araB}-lnt^{Ec}154-lacZ | Cm | This study |
| pCHAP6594 | pBAD33-p_{araB}-lnt^{Ec}190-lacZ | Cm | This study |
| pCHAP6595 | pBAD33-p_{araB}-lnf^{Ec}218-lacZ | Cm | This study |
| pCHAP6596 | pBAD33-p_{araB}-lnt^{Ec}476-lacZ | Cm | This study |
| pCHAP6597 | pBAD33-p_{araB}-lnt^{Ec}512-lacZ | Cm | This study |

Ap, ampicillin;
Cm, chloramphenicol;
Km, kanamycin.

TABLE 5

Primers.

| | |
|---|---|
| Name | Sequence (5′ to 3′) |

| | | |
|---|---|---|
| 5′-kan | GAATTCTACGTGCCCGGGAGATC | (SEQ ID NO: 6) |
| kan-3′ | TAATGACCCGGGCGTGATGCGGCCGC | (SEQ ID NO: 7) |
| 5′-para | TCTAGACTGTAACAAAGCGGGACC | (SEQ ID NO: 8) |
| para-3′ | CCCGTTTTTTTGGGCTAGGTCGAC | (SEQ ID NO: 9) |
| 5′-ybe-k | GTCAAAATCCCGGATGACTCACCCCAGCCGAAGCTGGATGAATAA | (SEQ ID NO: 10) |
| | GCGGCCGCATCACGCCCGG | |
| p-lnt-3′ | CCCGTTTTTTTGGGCTAGGTCGACCCGAAACTGGATAGATAACTAC | (SEQ ID NO: 11) |
| | ATGGCTTTTGCCTCATTAATTGAA | |
| 5′-cutE | GAATTCACCGAAACTGGATAGATAAC | (SEQ ID NO: 12) |
| cutE-3′ | CTGCGTCAGCGACGTAAACATCACCATCACCATCATTAAGGATCC | (SEQ ID NO: 13) |
| 5′-lnt | GAATTCGAATAACAGCAATAGTGGAC | (SEQ ID NO: 14) |
| lnt-3′ | TCTGCGACAACGACGGAAACATCACCATCACCATCATTAAGGATCC | (SEQ ID NO: 15) |
| 5′-cutE435 | GAATGCGTGCGCTGAAGCTGGCGCGCCCAC | (SEQ ID NO: 16) |
| 5′-phoA | CTGCAGCTCAGGGCGATATTACTGCACC | (SEQ ID NO: 17) |
| phoA-3′ | GCCGCTCTGGGGCTGAAATAATAAGCTT | (SEQ ID NO: 18) |
| 5′-lacZ | CTGCAGCCGTCGTTTTACAACGTCGTG | (SEQ ID NO: 19) |
| lacZ-3′ | TACCAGTTGGTCTGGTGTCAAAAATAAAAGCTT | (SEQ ID NO: 20) |
| 5′-intEc | TCTAGAACCGAAACTGGATAGATAAC | (SEQ ID NO: 21) |
| lntEc30-3′ | CGCTGGCCTTCTCTCCTGCAG | (SEQ ID NO: 22) |
| lntEc53-3′ | GACCTTTAACCGCCGTCCTGCAG | (SEQ ID NO: 23) |
| lntEc80-3′ | GGTCTATGTCAGCATCGCTGCAG | (SEQ ID NO: 24) |
| lntEc117-3′ | CTGTCGCGTCTGTGGCCTGCAG | (SEQ ID NO: 25) |
| lntEc154-3′ | GTGGTTACAGTTCGGCTATTCTGCAG | (SEQ ID NO: 26) |
| lntEc190-3′ | TGGCGTTGGTCAAACGCACTGCAG | (SEQ ID NO: 27) |
| lntEc218-3′ | AGTGGTTTACCCCACAACCTGCAG | (SEQ ID NO: 28) |
| lntEc476-3′ | TAACCACTAACGTGACGCCTGCAG | (SEQ ID NO: 29) |
| lntEc513-3′ | GAGTCTGCGTCAGCGACGTAAACCTGCAG | (SEQ ID NO: 30) |

TABLE 6
Complementation of lnt^{ts} mutation in
S. enterica and of p_{araB}-lnt mutation in E. coli.

| | | Growth temperature | | |
|---|---|---|---|---|
| Strain - plasmid | Protein produced | 30° C. | 37° C. | 42° C. |
| LT2 - pUC18 | / | ++ | ++ | + |
| LT2 - pCHAP6571 | LntEc | ++ | ++ | + |
| LT2 - pCHAP6573 | LntSe | ++ | + | + |
| LT2 - pCHAP6576 | Lnt^{Ec}(E435K) | ++ | + | − |
| LT2 - pCHAP6574 | LntSe(E435K) | + | + | − |
| SE5312 - pUC18 | / | ++ | − | − |
| SE5312 - pCHAP6571 | Lnt^{Ec} | ++ | ++ | + |
| SE5312 - pCHAP6573 | LntSe | ++ | ++ | ++ |
| SE5312 - pCHAP6576 | Lnt^{Ec}(E435K) | ++ | − | − |
| SE5312 - pCHAP6574 | LntSe(E435K) | ++ | − | − |

| | Protein produced | Ara | Glu | Ara | Glu | Ara | Glu |
|---|---|---|---|---|---|---|---|
| PAP8504 - pUC18 | / | ++ | − | ++ | − | + | − |
| PAP8504 - pCHAP6571 | Lnt^{Ec} | ++ | ++ | ++ | ++ | ++ | ++ |
| PAP8504 - pCHAP6573 | LntSe | ++ | ++ | ++ | ++ | + | ++ |
| PAP8504 - pCHAP6576 | Lnt^{Ec}(E435K) | ++ | ++ | ++ | ++ | ++ | ++ |
| PAP8504 - pCHAP6574 | LntSe(E435K) | ++ | ++ | + | − | + | − |

Strains LT2, SE5312 and PAP8504 (p_{araB}-lnt) producing Lnt^{Ec}, Lnt^{Se}, Lnt^{Se}(E435K) and Lnt^{Ec}(E435K) encoded by pCHAP6571, pCHAP6573, pCHAP6574 and pCHAP6576, respectively, or carrying the empty vector (pUC 18) were grown on LB agar containing 100 mM IPTG at 30° C., 37° C. and 42° C. PAP8504 derivatives were tested on LB agar containing glucose (Glu) or arabinose (Ara). After overnight incubation, colonies were scored as ++(normal size), + (small) or − (no colonies).

### REFERENCES

1. Madan Babu, M., and Sankaran, K. (2002) Bioinformatics 18, 641-643
2. Ichihara, S., Hussain, M., and Mizushima, M. (1981) J. Biol. Chem. 256, 3125-3129
3. Shu, W., Liu, J., Ji, H., and Lu, M. (2000) J. Mol. Biol. 299, 1101-1112
4. Braun, V. (1975) Biochim. Biophys. Acta 415, 335-377
5. Hirota, Y., Suzuki, H., Nishimura, Y., and Yasuda, S. (1977) Proc. Natl. Acad. Sci. USA 74, 1417-1420
6. Hantke, K., and Braun, V. (1973) Eur. J. Biochem. 34, 284-296
7. Wu, H. C. (1996) in Escherichia coli and Salmonella. Cellular and Molecular Biology (Neidhardt, F. C., Curtiss III, R., Ingraham, J. L., Low, K. B., Magasanik, B., Reznikoff, W. S., Riley, M., Schaechter, M., and Umbarger, H. E., eds) Vol. 1, pp. 1005-1014, 2 vols., ASM Press, Washington D.C. USA
8. Yakushi, T., Masuda, K., Narita, S.-i., Matsuyama, S.-i., and Tokuda, H. (2000) Nature Cell. Biol. 2, 212-218
9. Matsuyama, S., Tajima, T., and Tokuda, H. (1995) EMBO. J 14, 3365-3372
10. Matsuyama, S., Yokota, N., and Tokuda, H. (1997) EMBO. J 16, 6947-6955
11. Yamaguchi, K., and Inouye, M. (1988) J. Bacteriol 170, 3747-3749
12. Pugsley, A. P., and Kornacker, M. G. (1991) J. Biol. Chem. 266, 13640-13645
13. Yamaguchi, K., Yu, F., and Inouye, M. (1988) Cell 53, 423-432
14. Seydel, A., Gounon, P., and Pugsley, A. P. (1999) Mol. Microbiol. 34, 810-821
15. Gennity, J. M., Kim, H., and Inouye, M. (1992) J. Bacteriol. 174, 2095-2101
16. Terada, M., Kuroda, T., Matsuyama, S.-i., and Tokuda, H. (2001) J. Biol. Chem. 276, 47690-47694
17. Robichon, C., Bonhivers, M., and Pugsley, A. P. (2003) Mol. Microbiol. 49, 1145-1154.
18. Masuda, K., Matsuyama, S.-I., and Tokuda, H. (2002) Proc Natl Acad Sci USA 99, 7390-7395
19. Narita, S.-i., Kanamura, K., Matsuyama, S.-I., and Tokuda, H. (2003) Mol. Microbiol. 49, 167-177.
20. Hara, T., Matsuyama, S., and Tokuda, H. (2003) J. Biol. Chem. 279, 40408-40414
21. Gupta, S. D., and Wu, H. C. (1991) FEMS Microbiol. Lett. 78, 37-42
22. Gupta, S. D., Gan, K., Schmid, M. B., and Wu, H. C. (1993) J. Biol. Chem. 268, 16551-16556
23. Rogers, S. D., Bhave, M. R., Mercer, J. F. B., Camakaris, J., and Lee, B. T. O. (1991) J Bacteriol 173, 6742-6748
24. Tiwari, R. P., Reeve, W. G., Dilworth, M. J., and Glenn, A. R. (1996) Microbiology 142, 601-610.
25. Tjalsma, H., Kontinen, V. P., Pragai, Z., Wu, H., Meima, R., Venema, G., Bron, S., Sarvas, M., and Maarten van Dijl, J. (1999) J. Biol. Chem. 274, 1698-1707
26. Navarre, W. W., Daefler, S., and Schneewind, O. (1996) J. Bacteriol. 178, 441-446
27. Fukuda, A., Matsuyama, S.-I., Hara, T., Nakayama, J., Nagasawa, H., and Tokuda, H. (2002) J. Biol. Chem. 277, 43512-43518
28. Hayashi, S., Chang, S. Y., Chang, S., Giam, C. Z., and Wu, H. C. (1985) J. Biol. Chem. 260, 5753-5759.
29. Yakushi, T., Tajima, T., Matsuyama, S.-I., and Tokuda, H. (1997) J. Bacteriol. 179, 2857-2862
30. Datsenko, K. A., and Wanner, B. L. (2000) Proc. Natl. Acad. Sci. USA. 97, 6640-6645
31. Richet, E., and Søgaard-Andersen, L. (1994) EMBO J. 13, 4558-4567
32. Miller, V. L., and Mekalanos, J. J. (1988) J Bacteriol 170, 2575-2583
33. Spratt, B. G., Hedge, P. J., te Heesen, S., Edelman, A., and Broome-Smith, J. K. (1986) Gene 41, 337-342
34. Guzman, L. M., Belin, D., Carson, M. J., and Beckwith, J. (1995) J Bacteriol 177, 4121-4130
35. Miller, J. H. (1972) Experiments in Molecular Genetics., Cold Spring Harbor Laboratory, Cold Spring Harbor, N.Y. USA
36. Kumamoto, C. A., and Gannon, P. M. (1988) J Biol Chem 263, 11554-11558
37. Simons, R. W., Houman, F., and Kleckner, N. (1987) Gene 53, 85-96
38. Leduc, M., Ishidate, K., Shakibai, N., and Rothfield, L. (1992) J Bacteriol 174, 7982-7988
39. Lin, J. J., Kanazawa, H., Ozols, J., and Wu, H. C. (1978) Proc. Natl. Acad. Sci. USA. 75, 489-495.
40. Ichikawa, J. K., Chuan, L. I., Fu, J., and Clarke, S. (1994) J Bacteriol 176, 1630-1638
41. Manoil, C. (1990) J. Bacteriol. 172, 1035-1042
42. von Heijne, G. (1980) Biochem Biophys Res Commun 93, 82-86
43. Moreno, F., Fowler, A. V., Hall, M., Silhavy, T. J., Zabin, I., and Schwartz, M. (1980) Nature 286, 356-359
44. Hu, L. A., and King, S. C. (1998) Biochem. J. 336, 69-76
45. Huber, F., and Erni, B. (1996) Eur. J. Biochem. 239, 810-817.
46. Sarsero, J. P., and Pittard, A. J. (1995) J. Bacteriol. 177, 297-306.
47. Suzuki, M., Hara, H., and Matsumoto, K. (2002) J Bacteriol 184, 5418-5425
48. Tanake, K., Matsuyama, S.-I., and Tokuda, H. (2001) J. Bacteriol. 183, 6538-6542.
49. Tajima, T., Yokota, N., Matsuyama, S., and Tokuda, H. (1998) FEBS. Lett. 439, 51-54
50. Gupta, S. D., Wu, H. C., and Rick, P. D. (1997) J. Bacteriol. 179, 4477-4484.
51. Gupta, S. D., Lee, B. T., Camakaris, J., and Wu, H. C. (1995) J. Bacteriol. 177, 4207-4215.
52. Kawula, T. H., Spinola, S. M., Klapper, D. G., and Cannon, J. G. (1987) Mol. Microbiol. 1, 179-185
53. Reilly, T. J., Chance, D. L., and Smith, A. L. (1999) J. Bacteriol. 181, 6797-6805.
54. Brissette, J. L., Russel, M., Weiner, L., and Model, P. (1990) Phage shock protein, a stress protein of Escherichia coli. Proc. Natl. Acad. Sci. USA 87: 862-866.
55. Gupta, S. D., Gan, K., Schmid, M. B., and Wu, H. C. (1993) Characterization of a temperature-sensitive mutant of Salmonella typhimurium defective in Apolipoprotein N-Acyltransferase. J. Biol. Chem. 268: 16551-16556.
56. Jackowski, S., and Rock, C. O. (1986) Transfer of fatty acids from the 1-position of phosphatidylethanolamine to the major outer membrane lipoprotein of Escherichia coli. J Biol Chem 261: 11328-11333.
57. Kumaran, D., Eswaramoorthy, S., Gerchman, S. E., Kycia, H., Studier, F. W., and Swaminathan, S. (2003) Crystal structure of a putative CN hydrolase from yeast. Proteins 52: 283-291.
58. Nakai, T., Hasegawa, T., Yamashita, E., Yamamoto, M., Kumasaka, T., Ueki, T., Nanba, H., Ikenaka, Y., Takahashi, S., Sato, M., and Tsukihara, T. (2000) Crystal structure of N-carbamyl-D-amino acid amidohydrolase with a novel catalytic framework common to amidohydrolases. Structure Fold Des 8: 729-737.
59. Pace, H. C., Hodawadekar, S. C., Draganescu, A., Huang, J., Bieganowski, P., Pekarsky, Y., Croce, C. M., and Brenner, C. (2000) Crystal structure of the worm NitFhit Rosetta Stone protein reveals a Nit tetramer binding two Fhit dimers. Curr Biol 10: 907-917.
60. Raivio, T. L. (2005) Envelope stress responses and Gram-negative bacterial pathogenesis. Mol Microbiol 56: 1119-1128.
61. Robichon, C., Vidal-Ingigliardi, D., and Pugsley, A. P. (2005) Depletion of Apolipoprotein N-Acyltransferase Causes Mislocalization of Outer Membrane Lipoproteins in Escherichia coli. J Biol Chem 280: 974-983.
62. Wang, W. C., Hsu, W. H., Chien, F. T., and Chen, C. Y. (2001) Crystal structure and site-directed mutagenesis studies of N-carbamoyl-D-amino-acid amidohydrolase from Agrobacterium radiobacter reveals a homotetramer and insight into a catalytic cleft. J Mol Biol 306: 251-261.

1. An isolated or purified polypeptide comprising one Lnt periplasmic segment selected from the group consisting of sequences SEQ ID NO: 3, 4 and 5, sequences which consist essentially of amino acids residues 28-33, 76-87 or 212-488 of SEQ ID NO: 2, or a fragment thereof.

2. The isolated or purified polypeptide of claim 1 which consists of a fragment from 5 up to 250 amino acids residues.

3. The isolated or purified polypeptide according to claim 1, which comprises the Lnt periplasmic segment of SEQ ID NO: 3.

4. The isolated or purified polypeptide of claim 1, which comprises the Lnt periplasmic segment of SEQ ID NO: 4.

5. The isolated or purified polypeptide of claim 1, which comprises the Lnt periplasmic segment of SEQ ID NO: 5.

6. The isolated or purified polypeptide of claim 1 consisting essentially of residues 28-33, 76-87 or 212-488 of SEQ ID NO: 2.

7. A recombinant cell comprising a polynucleotide encoding at least one periplasmic segment of the Lnt protein according to claim 1, wherein said polynucleotide is under the control of an inducible promoter.

8. The cell of claim 7, which is a gram-negative cell.

9. The cell of claim 7, which is Escherichia coli.

10. The cell of claim 7, wherein the inducible promoter is an arabinose-AraC-dependent promoter.

11. The cell of claim 7, wherein said polynucleotide comprises an lnt gene.

12. The cell of claim 7, wherein said polynucleotide comprises SEQ ID NO: 1.

13. The cell of claim 7, which is E. coli strain PAP8504 deposited under accession number I-3310 at CNCM or E. coli strain PAP8505 deposited under accession number I-3311 at the CNCM.

14. The cell of claim 7, wherein said polynucleotide encodes residues 28-33, 76-87 or 212-488 of SEQ ID NO: 2 or a fragment thereof.

15. A method for identifying a test compound which binds to Lnt protein comprising contacting a compound with the polypeptide of claim 1.

16. The method of claim 15 further comprising determining the amount of binding of said compound to said polypeptide.

17. A method for identifying a compound which modulates the activity of Lnt protein comprising contacting a compound with a cell expressing a polypeptide comprising at least one periplasmic segment of the Lnt protein, and determining the amount of apolipoprotein which accumulates in the inner membrane and/or the amount of lipoprotein in the outer membrane and/or associated with the peptidoglycan.

18. A method for identifying a compound which modulates the activity of Lnt protein comprising: contacting a cell expressing a polypeptide comprising one periplasmic segment of the Lnt protein with a test compound and determining membrane integrity or cell viability compared to a cell not contacted with said test compound.

19. A method for identifying a bacterial gene coding for a protein functionally equivalent to Lnt, comprising introducing said bacterial gene into the cell of claim 7 in culture conditions where the inducible promoter is off, scoring the colonies and concluding that the bacterial gene encodes a protein functionally equivalent to Lnt if there are more colonies with the introduced bacterial gene than without.

20. The cell of claim 7, which is cell line PAP8504 (CNCM I-3310).

21. The cell of claim 7, which is cell line PAP8505 (CNCM I-3311).

22. The cell of claim 7, which is cell line PAP105 (pCHAP1441)(CNCM I-3312).

23. An isolated or purified polynucleotide encoding one or more Lnt protein domains described by FIG. 9 or by FIG. 10; the complement thereof; or a fragment thereof.

24. The isolated or purified polynucleotide of claim 23 which has a length from 15 up to 750 nucleotides.

25. A vector comprising the polynucleotide of claim 23.

26. A host cell comprising the polynucleotide of claim 23.

27. An isolated or purified polypeptide consisting essentially of one or more Lnt protein domains described by FIG. 9 or by FIG. 10 encoded by the polynucleotide of claim 23.

28. The method of claim 18, wherein Cpx and sigma E shock responses are used as cell viability determinants to identify a compound which inhibits the activity of Lnt protein.

29. The method of claim 19, wherein said bacterial gene is from a Gram-negative bacterium.

30. An isolated or purified polynucleotide encoding the polypeptide of claim 1.

31. The polynucleotide of claim 30, wherein it has a sequence included in SEQ ID NO: 1 or it has a sequence which is able to hybridize under stringent conditions to SEQ ID NO: 1 or its complement and which encodes a polypeptide having N-acyltransferase activity.

32. A technical platform comprising a least standard quantities of purified polypeptide of claim 1, reagents for testing the N-acyltransferase activity activity, and reagents for testing the amount of apolipoprotein which accumulates in the inner membrane and/or the amount of lipoprotein in the outer membrane and/or associated with the peptidoglycan.

33. A technical platform comprising at least a recombinant cell according to claim 7 or a cell selected from the group consisting of PAP8504 (CNCM I-3310), PAP8505 (CNCM I-3311) and PAP105 (pCHAP1441)(CNCM I-3312) reagents for testing the N-acyltransferase activity activity, and reagents for testing the amount of apolipoprotein which accumulates in the inner membrane and/or the amount of lipoprotein in the outer membrane and/or associated with the peptidoglycan.
